(19) United States
(12) Patent Application Publication
(10) Pub. No.: US 2015/0079047 A1
(43) Pub. Date: Mar. 19, 2015

(54) OCULAR THERAPEUTICS USING EMBRYONIC STEM CELL MICROVESICLES

(71) Applicant: THE REGENTS OF THE UNIVERSITY OF CALIFORNIA, Oakland, CA (US)

(72) Inventors: Debora B. Farber, Beverly Hills, CA (US); Steven D. Schwartz, Los Angeles, CA (US); Diana Katsman, Sherman Oaks, CA (US)

(21) Appl. No.: 14/394,688

(22) PCT Filed: Mar. 13, 2013
(86) PCT No.: PCT/US13/30733

§ 371 Date: Oct. 15, 2014

Related U.S. Application Data
(60) Provisional application No. 61/624,701, filed on Apr. 16, 2012.

(51) Int. Cl. C12N 5/0735; C12N 5/079; A61K 35/54

(52) U.S. Cl. CPC C12N 5/0606; A61K 35/545; C12N 5/0621
USPC 424/93.7; 435/366; 435/377; 435/29; 435/325

(57) ABSTRACT

Disclosed is a therapeutic composition comprising human embryonic stem cell-derived micro vesicles, and methods of their use, including treatment of eye pathologies and of obtaining retinal neural cells and retinal stem cells.

### CROSS-REFERENCE TO RELATED APPLICATION

This application claims the benefit of U.S. Provisional Patent Application Ser. No. 61/624,701 entitled “Ocular Therapeutics Using Stem Cell Microvesicles” which was filed Apr. 16, 2012. The entirety of the aforementioned application is herein incorporated by reference.

### FIELD OF THE INVENTION

This invention is in the field of ophthalmological medicine, and more particularly, in the field of treatment for retinal degenerative and dystrophic diseases, optic nerve degenerations, anterior segment of the eye disease, such as cataract, corneal and dry eye disease, and systemic and local autoimmune diseases that adversely affect the eye.

### BACKGROUND OF THE INVENTION

Ocular diseases, such as retinal degeneration and dystrophies, are among the leading cause of irreversible blindness in the world; millions of people are affected with diabetic retinopathy, and various forms of macular degeneration, such as age-related macular degeneration and other hereditary retinal and macular degenerations. Cataract, glaucoma, corneal and dry eye conditions represent the majority of global, non-retinal blinding conditions. Therapies to slow down ocular disease and augment repair and or regeneration of tissue, for example, by improving the regenerative capacity of ocular tissue, such as the retina, are in dire need.

### SUMMARY OF THE INVENTION

The present disclosure relates to a therapeutic fraction of embryonic stem cell (ESC)-derived microvesicles (ESMVs) and its therapeutic use in various compartments of the eye for treatment of ocular diseases and disorders.

It has been discovered how stem cells influence their environment in the eye. This discovery has been utilized to develop the present treatments which do not involve transplanting stem cells, but rather involves harvesting their regenerative signal and treating the afflicted ocular tissue with the regenerative signal alone. The disclosure provides a method of isolating such a therapeutic fraction and of identifying its selection. It further provides methods of initiating regeneration in the eye by inducing endogenous progenitor cells, such as Müller and microglial cells, inducing de-differentiation of retinal cell lines to a more retinal stem cell phenotype, facilitating re-differentiation of certain ocular cells, as well as methods of treating ocular diseases and disorders.

In one aspect, a method of obtaining retinal neural cells is provided, which comprises treating ocular neural progenitor cells with an amount of an ESMV fraction effective to cause the ocular neural progenitor cells to differentiate into retinal neural cells. In some embodiments, the differentiation of retinal progenitor cells or of microglial and/or Müller cells is measured by the presence of glutamine synthetase, Gad67, NeuN, Brn3a, and Syntaxin 1a in the treated cells.

In another aspect, the disclosure provides a method of obtaining cells with a retinal stem cell phenotype, comprising treating microglial cells and Müller cells for at least 8 hours with an effective amount of an embryonic stem cell-derived microvesicle fraction, and measuring the level of epidermal growth factor receptor (EGFR) in the treated cells, the level of EGFR in cells with a retinal stem cell phenotype being decreased relative to the level of EGFR in untreated microglial cells and Müller cells. In one embodiment, the ESMV fraction comprises human ESMVs.

In yet another aspect, a method of treating an eye pathology in a mammal is provided, comprising administering to the eye of the mammal a therapeutically effective amount of an embryonic stem cell-derived microvesicle (ESMV) fraction. In some embodiments, the ESMV fraction is administered by intravitreal injection, subretinal injection, interocular injection, or by topical administration. In certain embodiments, the ESMV fraction is administered by continuous or bolus release.

In specific embodiments, administration may be provided by a device, such as a contact lens or device including a pump. In some embodiments, the form of administration is by continuous or bolus release.

In certain embodiments, the ESMV fraction comprises human ESMVs.

In some embodiments, the eye pathology treated is age-related macular degeneration, myopic degeneration, diabetic retinopathy, glaucoma, the retinitis pigmentosa complex, inherited retinal degeneration, uveitis, dry eye, optic neuropathy, corneal or anterior segment ocular diseases, such as, but not limited to, ocular cicatricial pemphigoid, benign and malignant Mooren's corneal ulcer, or rheumatoid arthritis.

In certain embodiments, the eye pathology is glaucoma and the ESMV fraction is administered topically, intraocularly, or by intravitreal injection. In other embodiments, the eye pathology is age-related macular degeneration (AMD) or photoreceptor/RPE degeneration, and the ESMC fraction is administered by intravitreal, intraocular, or subretinal injection. In yet other embodiments, the eye pathology is retinal degeneration, and the ESMV fraction is administered by subretinal injection. In still other embodiments, the eye pathology is dry eye, corneal disease, or anterior segment ocular disease, and the ESMV fraction is administered by topical application.

In particular embodiments, the mammal being treated is human, and the ESMV fraction comprises human ESMVs.

The disclosure also provides a therapeutic composition comprising human embryonic stem cells in an amount effective to cause ocular neural progenitor cells to regenerate. In some embodiments, the ocular neural progenitor cells are retinal progenitor cells. In specific embodiments, the ocular neural progenitor cells are microglial cells and/or Müller cells.

### DETAILED DESCRIPTION OF THE FIGURES

The foregoing and other objects of the present disclosure, the various features thereof, as well as the invention itself may be more fully understood from the following description, when read together with the accompanying drawings in which:

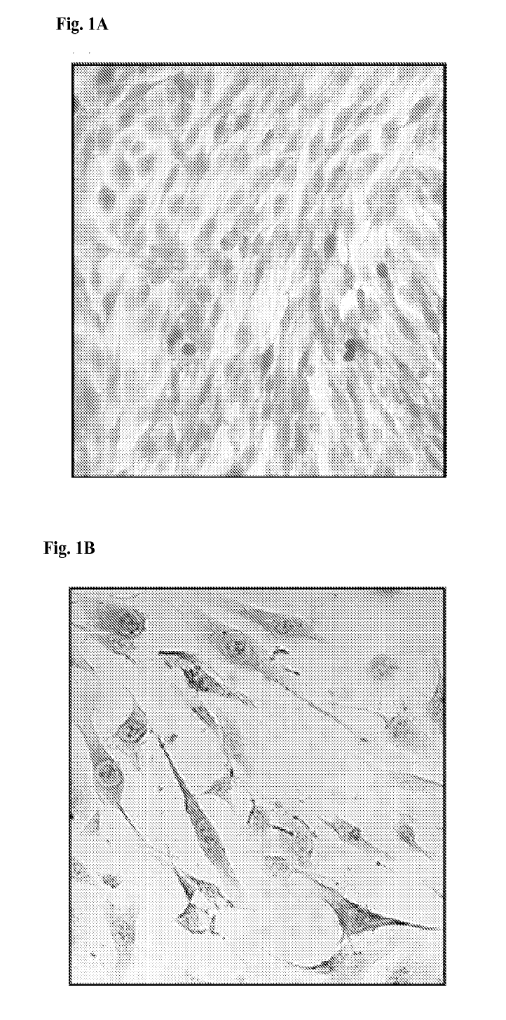

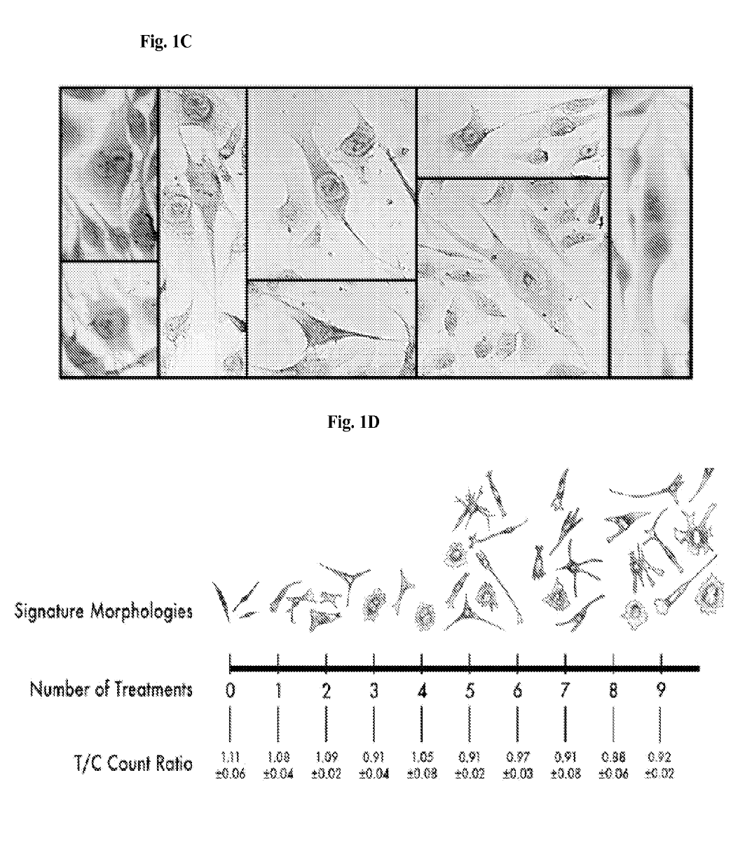

FIG. 1A is a microscopic representation showing untreated Müller cells growing as homogeneous, bipolar, spindle-like adherent cell “sheets,” where ESMV-treated (T) and control (C) cells were counted after each treatment and the ratio of treated to control cells calculated (T/C 6 S.E.M.);

FIG. 1B is a microscopic representation showing Müller cells post 9 ESMV treatments, growing as morphologically heterogeneous individual cells, some with multiple cellular processes, others with enlarged nuclei or multinucleated, many having visible metaphase plates and numerous stellar-shaped, where ESMV-treated (T) and control (C) cells were counted after each treatment and the ratio of treated to control cells calculated (T/C 6 S.E.M.);

FIG. 1C is a microscopic representation showing a collage of individual cells morphologically unique to the ESMV treatment group, where ESMV-treated (T) and control (C) cells were counted after each treatment and the ratio of treated to control cells calculated (T/C 6 S.E.M.);

FIG. 1D is a schematic representation of a timeline of the morphological changes that take place in Müller cells after ESMV treatments;

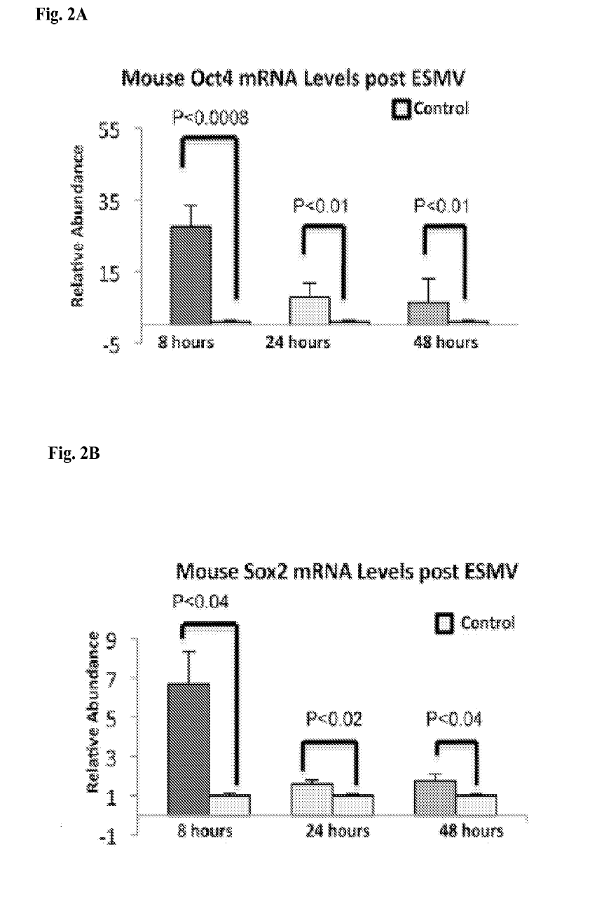

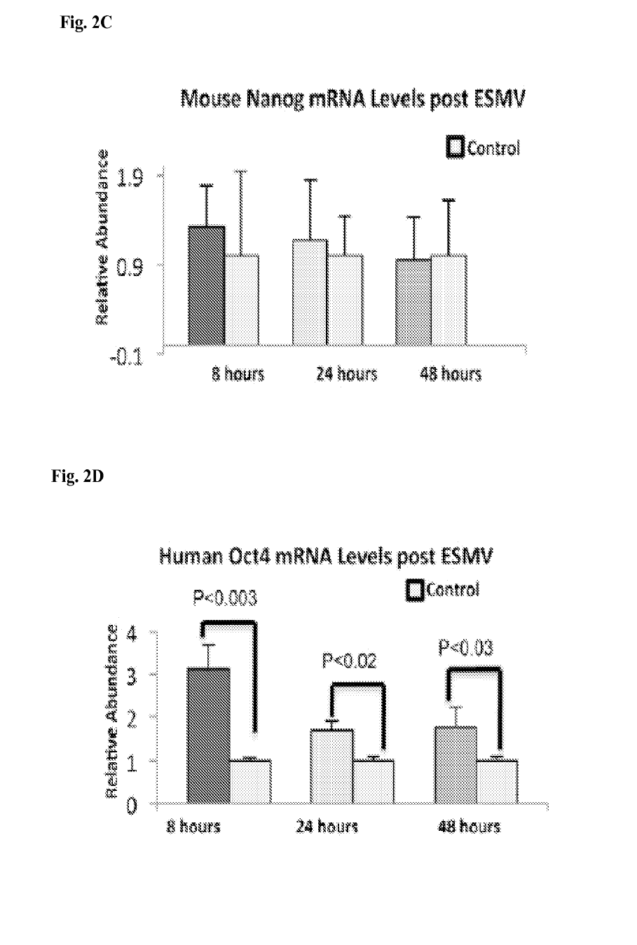

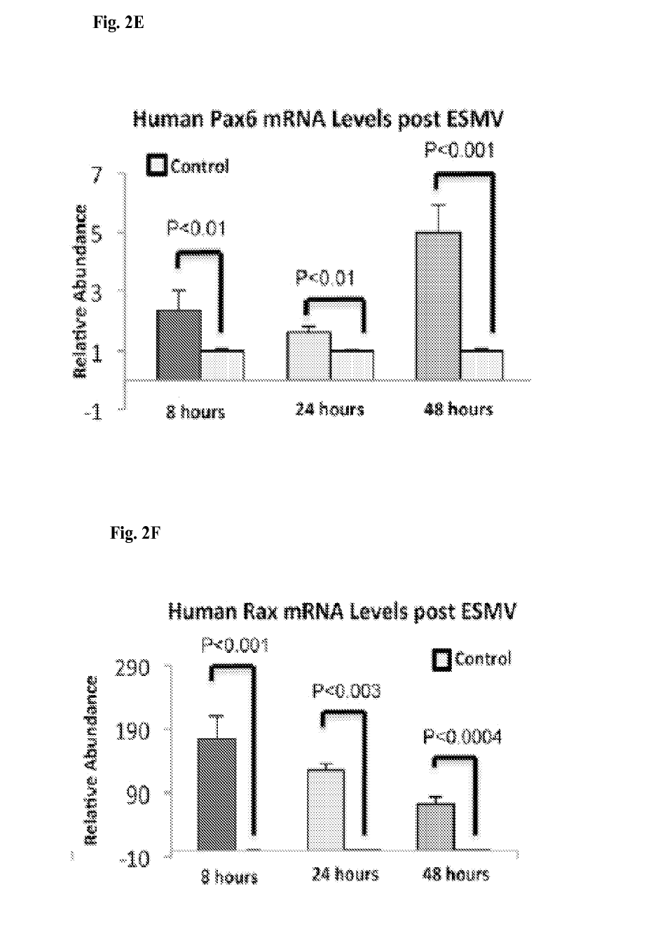

FIG. 2A is a graphic representation showing the fold change in expression for the embryonic stem cell-specific mouse Oct4 mRNA after ESMV treatment for 8 hours, 24 hours, and 48 hours relative to control (medium only), where Gapdh was used as a loading control for qRT-PCR and error bars represent S.E.M. Student's t-test, performed to assay the difference between experimental and control Müller cells groups, with p-values <0.05, except for Nanog mRNA;

FIG. 2B is a graphic representation showing the fold change in expression for the embryonic stem cell-specific mouse Sox2 mRNA after ESMV treatment for 8 hours, 24 hours, and 48 hours relative to control (medium only), where Gapdh was used as a loading control for qRT-PCR, and error bars represent S.E.M. Student's t-test, performed to assay the difference between experimental and control Müller cells groups, with p-values <0.05, except for Nanog mRNA;

FIG. 2C is a graphic representation showing the fold change in expression for the embryonic stem cell-specific mouse Nanog mRNA after ESMV treatment for 8 hours, 24 hours, and 48 hours relative to control (medium only), where Gapdh was used as a loading control for qRT-PCR, and error bars represent S.E.M. Student's t-test, performed to assay the difference between experimental and control Müller cells groups, with p-values <0.05, except for Nanog mRNA;

FIG. 2D is a graphic representation showing the fold change in expression for the embryonic stem cell-specific human Oct4 mRNA after ESMV treatment for 8 hours, 24 hours, and 48 hours relative to control (medium only), where Gapdh was used as a loading control for qRT-PCR, and error bars represent S.E.M. Student's t-test, performed to assay the difference between experimental and control Müller cells groups, with p-values <0.05, except for Nanog mRNA;

FIG. 2E is a graphic representation showing the fold change in expression for the embryonic stem cell-specific human Pax6 mRNA after ESMV treatment for 8 hours, 24 hours, and 48 hours relative to control (medium only), where Gapdh was used as a loading control for qRT-PCR, and error bars represent S.E.M. Student's t-test, performed to assay the difference between experimental and control Müller cells groups, with p-values <0.05, except for Nanog mRNA;

FIG. 2F is a graphic representation showing the fold change in expression for the embryonic stem cell-specific human Rax mRNA after ESMV treatment for 8 hours, 24 hours, and 48 hours relative to control (medium only), where Gapdh was used as a loading control for qRT-PCR, and error bars represent S.E.M. Student's t-test, performed to assay the difference between experimental and control Müller cells groups, with p-values <0.05, except for Nanog mRNA;

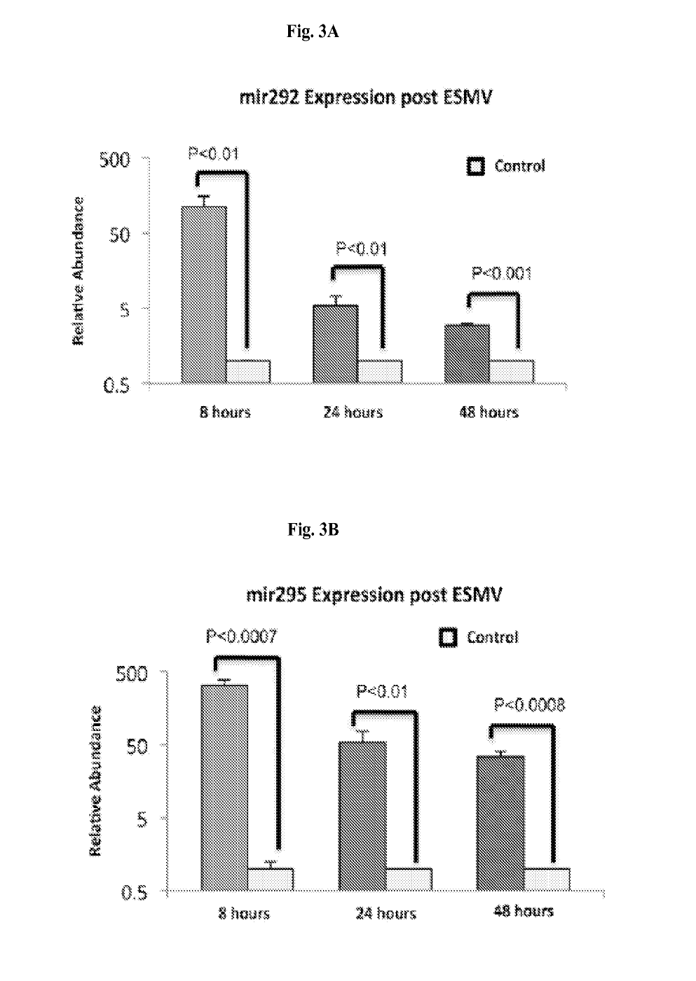

FIG. 3A is a graphic representation showing the fold change in the levels of ESC-specific miRNA 292 in Müller cells at 8 hours, 24 hours, and 48 hours after ESMV treatment relative to control, where the y-axis correspond to the fold changes between the treatment and control (light green) groups of Müller cells, and error bars represent S.E.M.; significant differences between experimental and control groups were determined by Student's t-test, where all p-values were <0.01;

FIG. 3B is a graphic representation showing the fold change in the levels of ESC-specific miRNA 295 in Müller cells at 8 hours, 24 hours, and 48 hours after ESMV treatment relative to control, where the y-axis correspond to the fold changes between the treatment and control (light green) groups of Müller cells, and error bars represent S.E.M.; significant differences between experimental and control groups were determined by Student's t-test, where all p-values were <0.01;

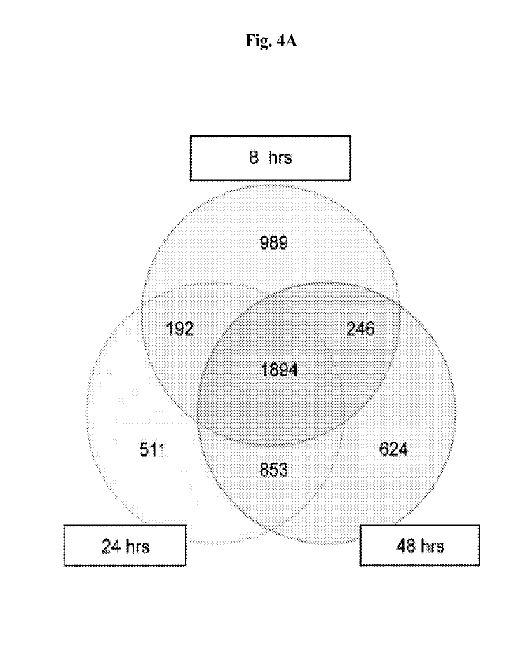

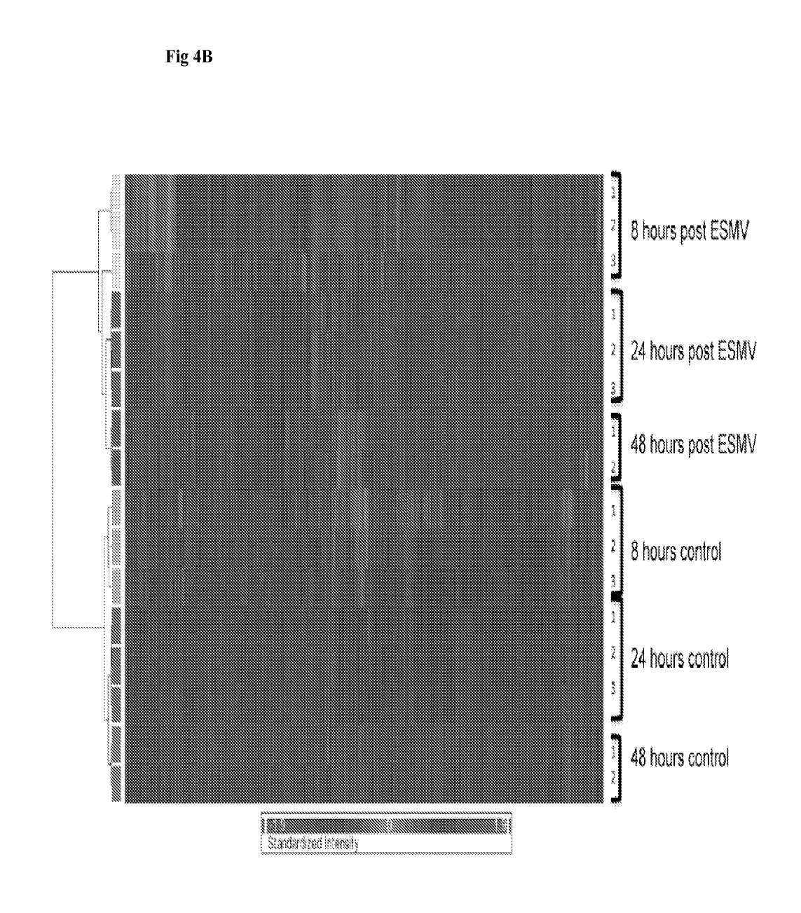

FIG. 4A is a diagrammatic representation of a Venn diagram of gene expression changes, as measured by microarray, in ESMV-treated versus control Müller cells at 8 hours, 24 hours, and 48 hours post-ESMV exposure;

FIG. 4B a diagrammatic representation of a heat map of hierarchal clustering of 16 samples based on the 1894 probes found to be differentially regulated in the Müller cells post-ESMV treatment versus control (p-value, 0.001 and a minimum of 3-fold difference in expression), with red representing up- and blue representing down-regulation; rows represent the samples and columns represent the genes;

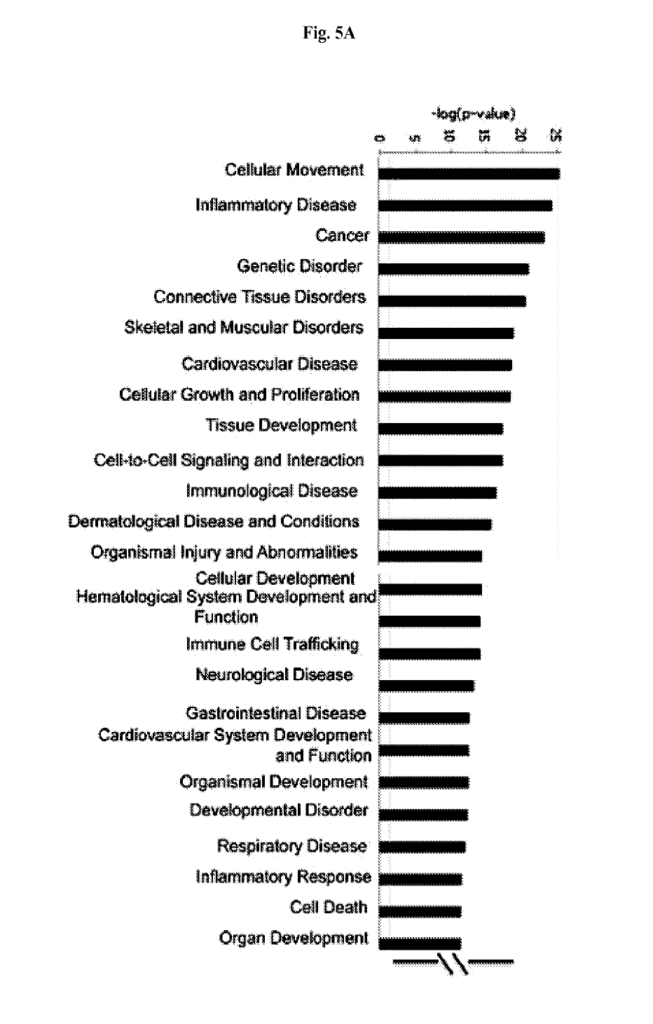

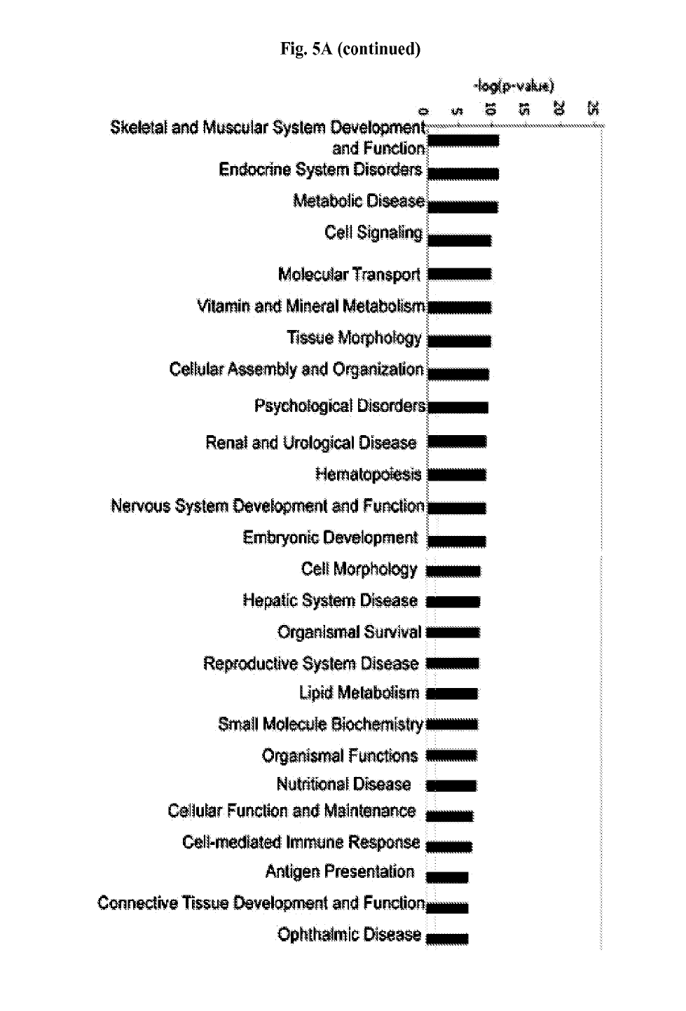

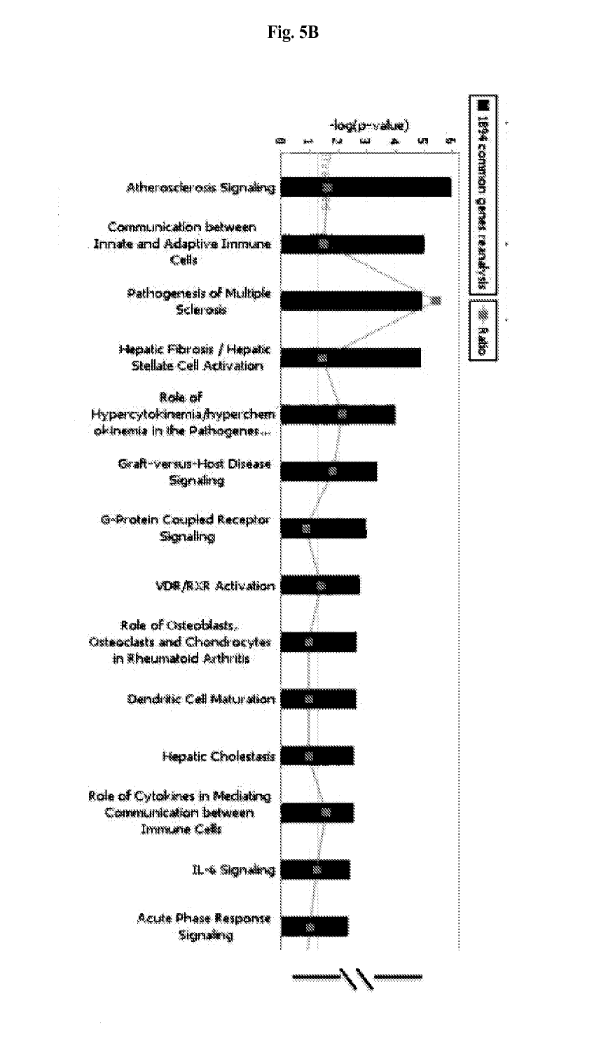

FIG. 5A is a graphic representation of an ingenuity pathway analysis of 1894 genes differentially regulated at all tested time points between ESMV-treated and control Müller cells at p<0.001 level and fold change ≧3 in specific function, where genes were tested for significant associated in specific cell functional signaling pathways versus random change association in a total curated database of gene interactions of over significant canonical pathways;

FIG. 5B is graphic representation of an ingenuity pathway analysis of 1894 genes differentially regulated at all tested time points between ESMV-treated and control Müller cells at p<0.001 level and fold change ≧3 in cell canonical signaling pathway, where genes were tested for significant associated in specific cell canonical signaling pathways versus random change association in a total curated database of gene interactions of over significant canonical pathways;

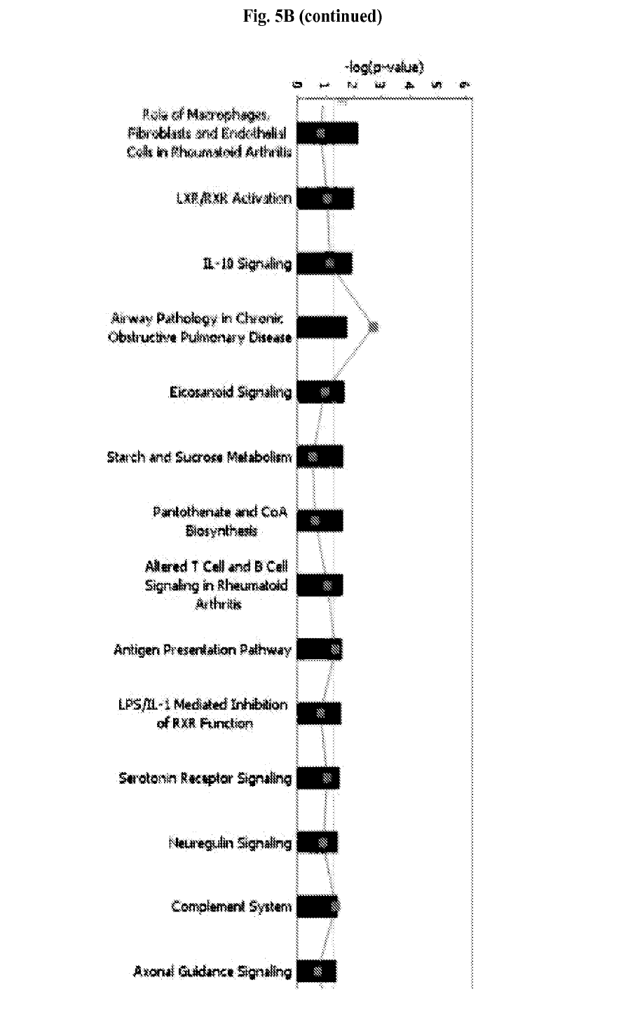

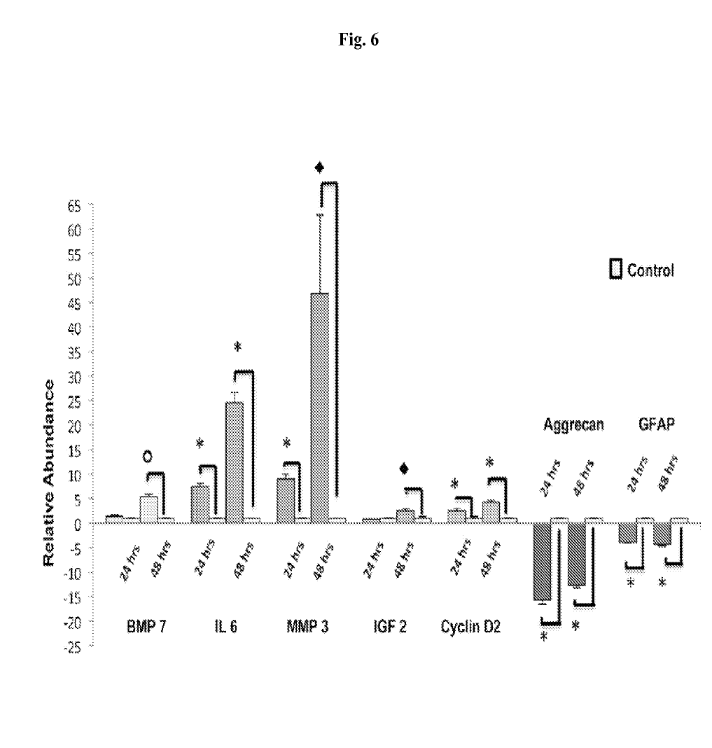

FIG. 6 is a graphic representation of an qRT-PCR analysis showing gene expression changes of microarray-identified genes in Müller cells at 24 hours and 48 hours post-ESMV treatment compared to untreated controls, where each bar represents the relative abundance of the genes tested in ESMV-treated versus untreated Müller cells and error bars represent S.E.M.;

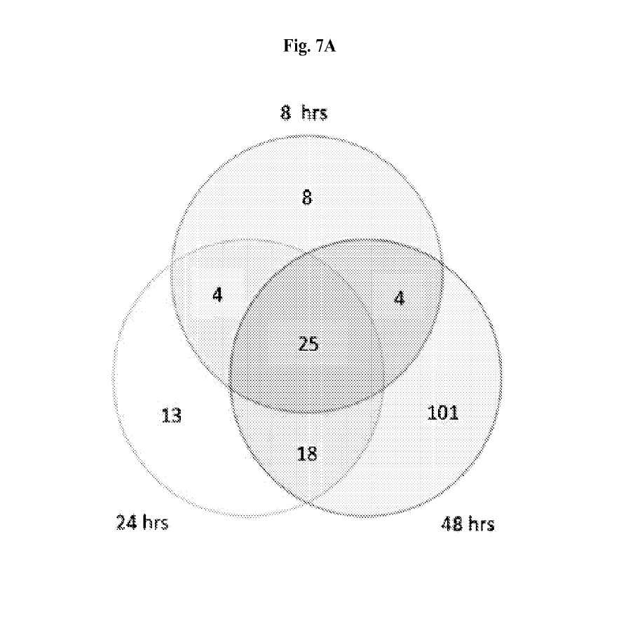

FIG. 7A is a graphic representation of a Venn diagram of miRNA expression changes in ESMV-treated versus control Müller cells at 8 hours, 24 hours, and 48 hours post-ESMV treatment;

FIG. 7B is a graphic representations of a heat map of hierarchal clustering of 16 samples based on 25 miRNA probes differentially regulated in ESMV-treated versus control Müller cells at all times tested. (p<0.05, minimum 3-fold difference in expression), where each row represents a single sample, and each column-a single miRNA, and where the red or blue color represents relatively high or low expression, respectively;

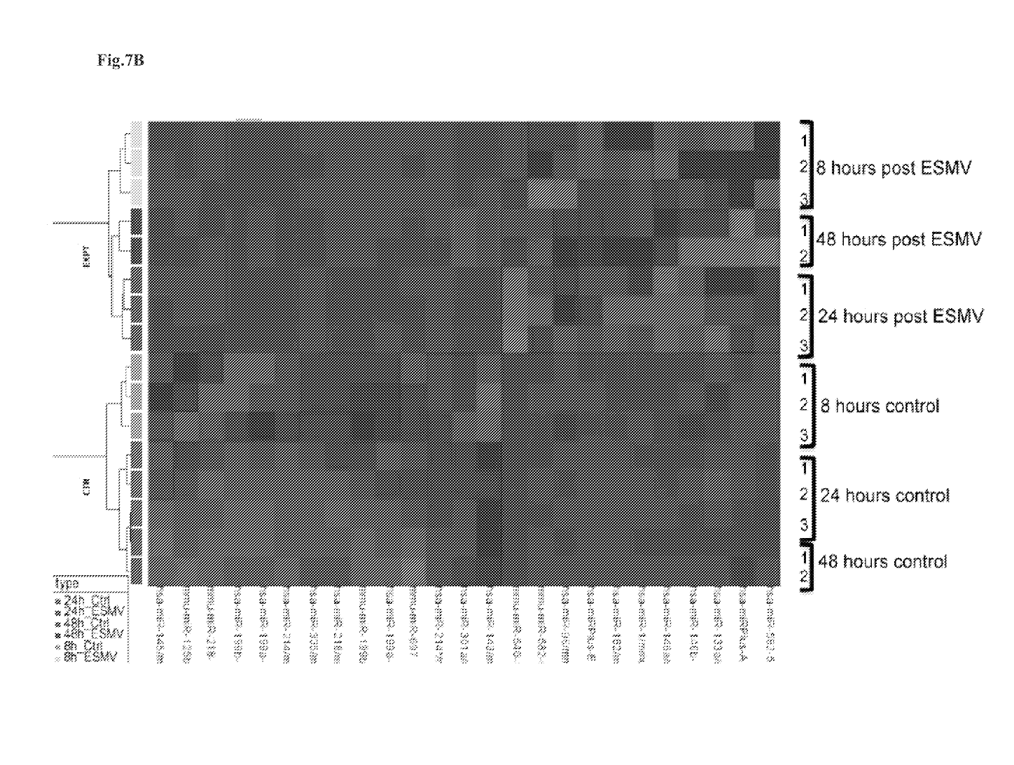

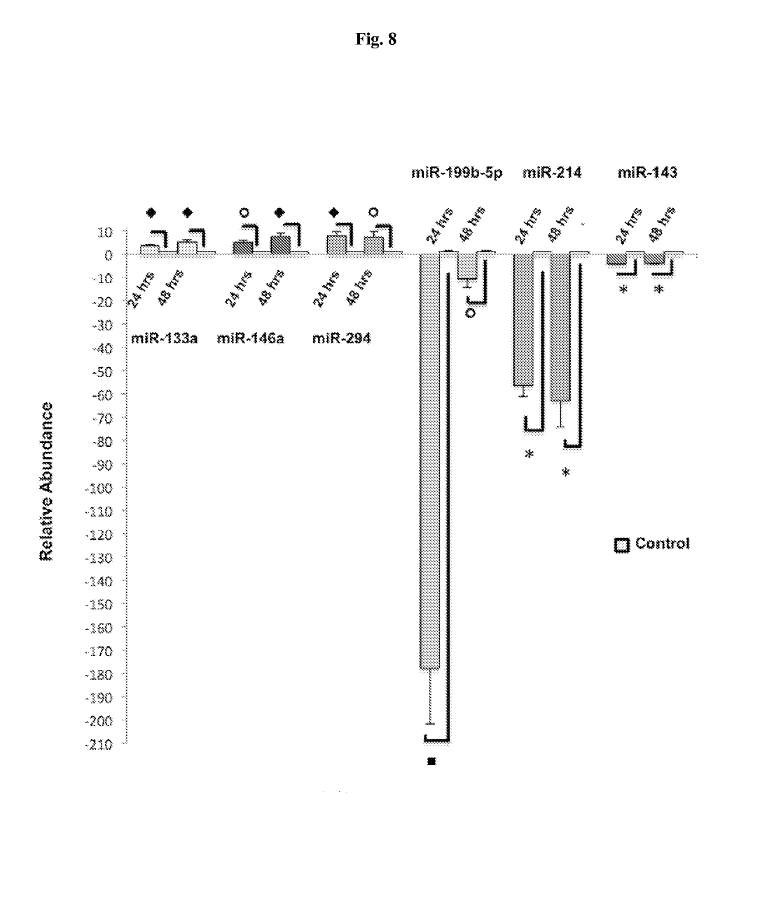

FIG. 8 is a graphic representation showing qRT-PCR analysis of select miRNAs involved maintenance of pluripotency, de-differentiation, cell fate determination and differentiation, in ESMV-treated versus control Müller cells, where each bar represents the relative abundance of the miRNAs tested in ESMV-treated Müller cells versus untreated control cells and error bars represent SEM;

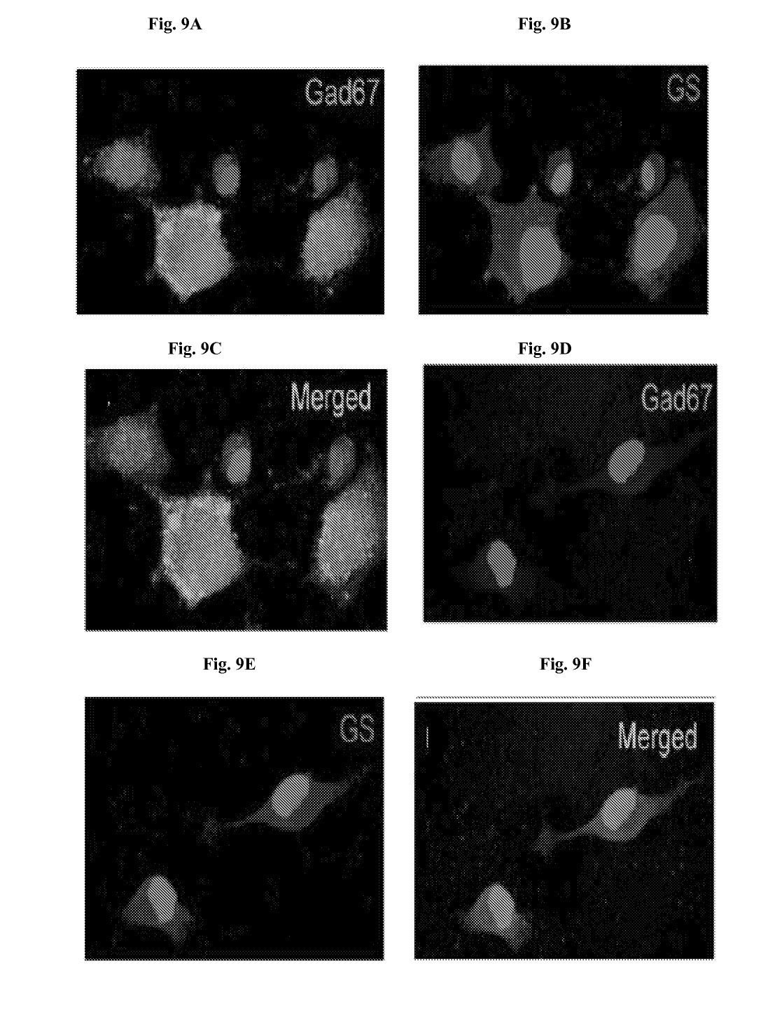

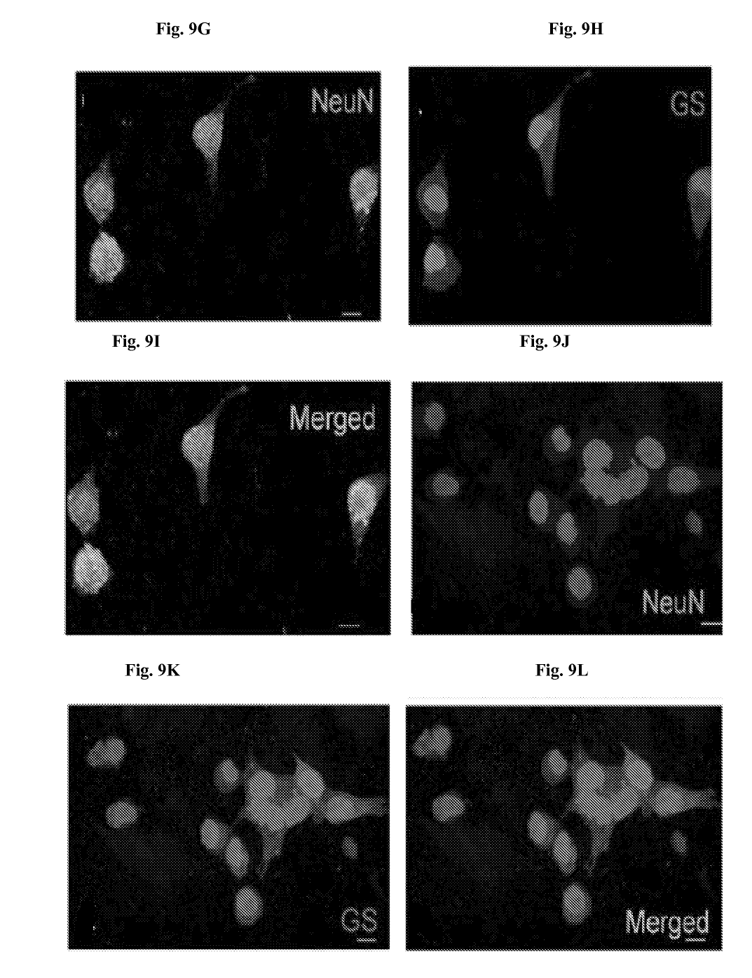

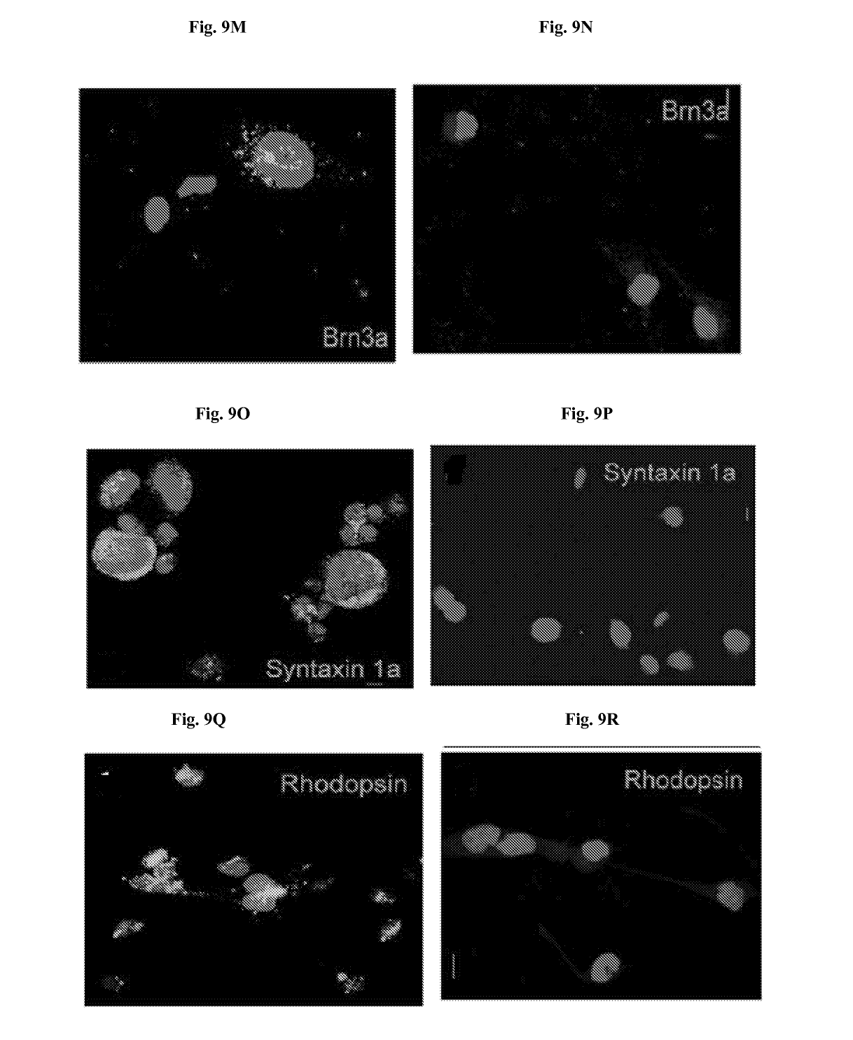

FIGS. 9A-9R are representations of confocal photomicrographs showing ESMV-treated and control Müller cells immunostained for markers of various retinal lineages, where Figs. A-L show cells were double stained with Gad67 (amacrine and horizontal cells; green) or NeuN (amacrine and ganglion cells; green) and the marker of Müller cells, glutamine synthetase (red); where FIGS. 9A-9C show Gad67-stained ESMV-treated Müller cells; where FIGS. 9D-9F show Gad67-stained control Müller cells; where FIGS. 9G-9I show NeuN-stained ESMV-treated Müller cells; where FIGS. 9J-9L show NeuN-stained control Müller cells; where the third panel of each row shows the merged first two images; where FIG. 9M shows ESMV-treated and FIG. 9N shows control Müller cells, stained for Brn3a (green), a marker of retinal ganglion cells; FIG. 9O shows ESMV-treated and FIG. 9P shows control Müller cells stained for Syntaxin 1a (green), a marker of amacrine cells; FIG. 9Q shows ESMV-treated and FIG. 9R shows control Müller cells stained for rhodopsin (green), a marker of rod photoreceptors, where cell nuclei were labeled with 496-diamidino-2-phenylindole (DAPI, blue), and scale bar 10 mm for all panels with images showing z-axis projections of 1561 mm in all channels;

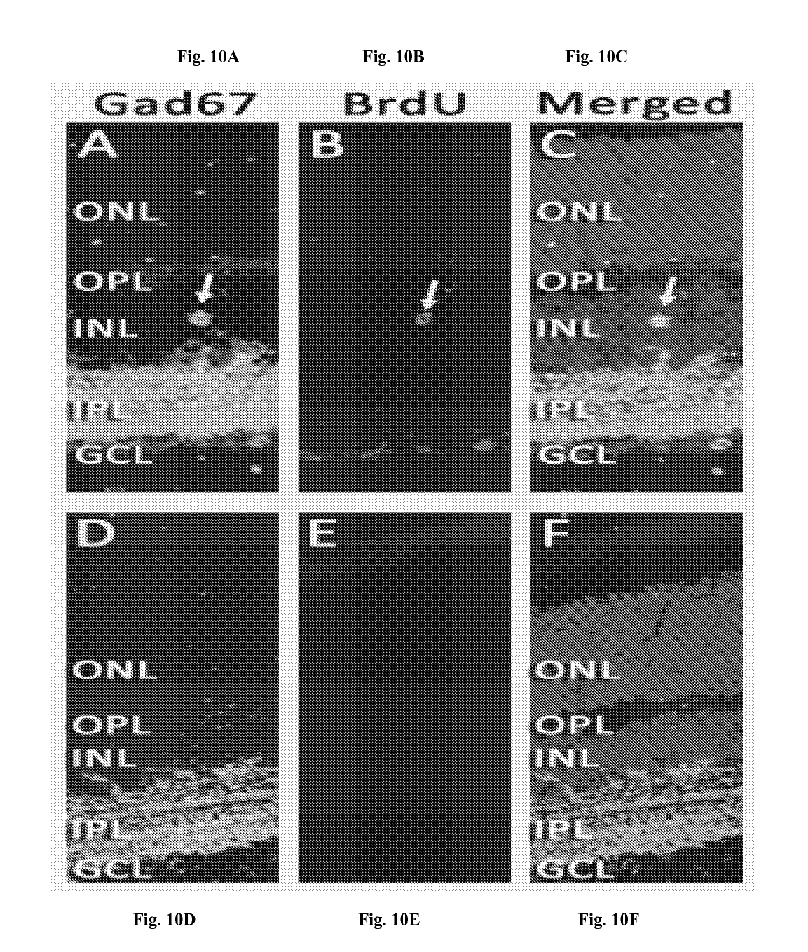

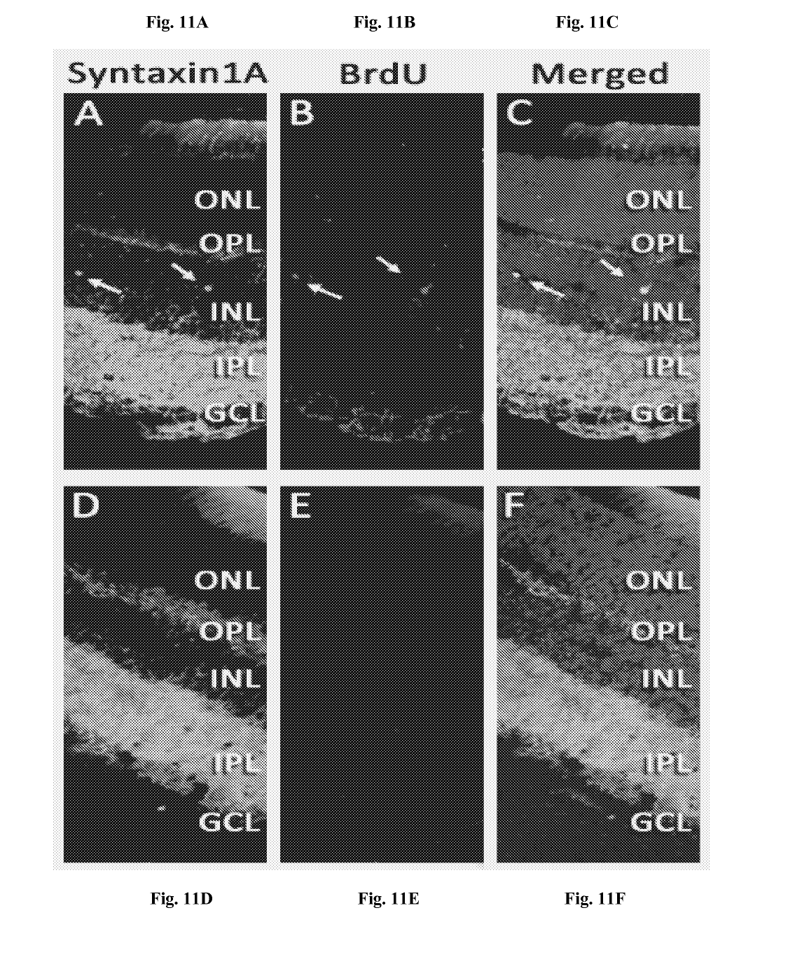

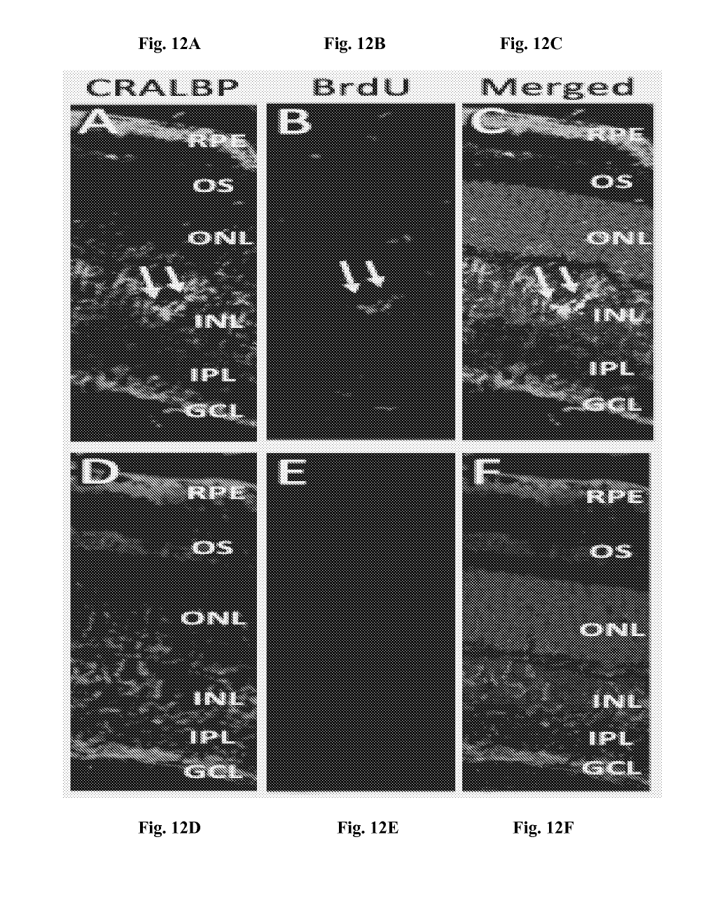

FIGS. 10A-10F are representations of confocal micrographs showing Gad67 expression (FIGS. 10A and 10C), BrdU expression (FIGS. 10B and 100D), and merged expression (FIGS. 10C and 10D) seven days (FIGS. 10A-10C) and 30 days (FIGS. 10D-10F) post-injection;

FIGS. 11A-11F are representations of confocal micrographs showing Syntaxin 1a expression (FIGS. 11A and 11C), BrdU expression (FIGS. 11B and 11D), and merged expression (FIGS. 11C and 11D) seven days (FIGS. 11A-11C) and 30 days (FIGS. 11D-11F) post-injection;

FIGS. 12A-12F are representations of confocal micrographs showing CRALBP expression (FIGS. 12A and 12C), BrdU expression (FIGS. 12B and 12D), and merged expression (FIGS. 12C and 12D) seven days (FIGS. 12A-12C) and 30 days (FIGS. 12D-12F) post-injection; and

FIG. 13 is a representation of a scotopic dark-adapted ERG tracing of one of the animals improved post-ESMV treatment at arbitrarily chosen stimulus intensity of 0.05345 cd/m2, where the maximum wave amplitude in the untreated right eye (red) remained at approximately 330 μV, while the maximum wave amplitude improved to over 450 μV in the ESMV-treated left eye (blue).

### DETAILED DESCRIPTION

Throughout this application, various patents, patent applications, and publications are referenced. The disclosures of these patents, patent applications, and publications in their entireties are hereby incorporated by reference into this application in order to more fully describe the state of the art as known to those skilled therein as of the date of the invention described and claimed herein. The instant disclosure will govern in the instance that there is any inconsistency between the patents, patent applications, and publications and this disclosure.

The present disclosure provides a therapeutic composition comprising a fraction of human embryonic stem cell (ESC) microvesicles (ESMVs), the isolation and identification of their therapeutic fraction, and the application of this fraction as a therapeutic modality to treat the majority of the diseases that afflict the eye.

Embryonic stem cells (ESCs) are known to release a population of microvesicles (ESMVs) heterogeneous in size (30 nm to 1 μm) into the extracellular environment (Ratajczak et al. (2006) Leukemia, 20:1487-1495). These microvesicles have the ability to transfer their contents to cells of other origins (Yuan et al. (2009) PLoS ONE, 4 (3); e4722:1-8). ESMVs are enriched in mRNAs for early transcription factors and miRNAs important for stem cell pluripotency. mRNAs that are important for maintenance of ESC pluripotency are abundant in ESMVs, as are miRNAs, small non-coding RNA molecules that play a pivotal role in maintenance of stem cell pluripotency and cell fate determination of most cells.

Müller cells, like microglial cells, are retinal progenitor cells as they have the ability to differentiate along multiple retinal lineages, such as photoreceptors and inner retina neurons.

It has been discovered that ESMVs can transfer their internal contents of stem cell mRNA, miRNA, and protein to cultured human retinal progenitor cells (Müller cells and microglial cells), thereby inducing the activation of endogenous, adult, quiescent progenitor cells in damaged tissue. Although not meant to be limited by any particular theory, this transfer is believed to occur in part through the merging of the ESMV membrane with other cell membranes.

It has been determined that ESMVs added to the cultures of retinal Müller cells induced morphological changes towards a more de-differentiated progenitor phenotype (FIG. 1). This ESMV fraction selectively transfers ESC mRNA and miRNA, resulting in induction of embryonic and early retinal genes in Müller cells. In addition, ESMV treatment of Müller cells induced a transcriptome change, indicative of de-differentiation and activation of a retinal re-generation program. Treatment of Müller cells resulted in the up-regulation of pluripotency and early retinal genes, genes involved in retinal protection and inducers of retinal regeneration, as well as multiple extracellular matrix (ECM)-modifying molecules that create a permissive environment for retinal regeneration. ESMV treatment also resulted in the down-regulation of genes promoting differentiation and inhibitory ECM and scar components. Moreover, ESMVs induced a shift in the miRNA transcriptome of Müller cells towards a de-differentiated progenitor state. These results demonstrate that ESMVs are therapeutic agents which can activate the retina's endogenous regenerative potential.

Thus, by their transfer ability, ESMVs are able to induce morphological changes in cultured retinal progenitor cells towards a more de-differentiated phenotype and also to initiate the regeneration of retinal tissue.

Cultured Müller cells exposed to ESMVs can up-regulate genes related to pluripotency (Oct4, Lin28, Klf4, and LIF), upregulate early retinal genes (BMP7, Pax6, and Rax), upregulate genes involved in retinal protection (IL6, CSF2), and regeneration (FGF2, IGF2, GDNF), and upregulate extracellular matrix-modifying genes known to create permissive environment for tissue remodeling (e.g., MMP3). In contrast, ESMVs can down-regulate genes promoting differentiation (e.g., DNMT3a and GATA4). Müller cells are activated in the injured retina with some regenerative success. However, functional retinal recovery has heretofore not been accomplished.

ESMVs improve the functionality of damaged retinas of mouse models of retinal degeneration. ESMVs stimulate regeneration, at least in part, by inducing endogenous retinal progenitor cells to repopulate and repair damaged retina. After ESMV application, a significant (70%) improvement in the a and b waves of mouse ERG, as well as immunohistochemical evidence of retinal cell repopulation, have been found.

The advantage of using hESMVs versus ESCs for therapeutic applications to humans is that hESMVs are not cells, do not actively produce surface molecules, and are less likely to cause rejection and tumor formation. hESMV preparations used are free of endotoxin, non-immunogenic, non-tumorigenic, and contaminant free. The use of hESMVs avoids the possible long-term maldifferentiation of engrafted intact ESCs and eliminates the risk of their malignant transformation.

The present disclosure demonstrates that ESMVs can also specifically stimulate or initiate regeneration of ocular compartments for regeneration and repair of damage. Examples of damage to be repaired by ESMVs actuating intrinsic regenerative agents range from corneal abrasion, ulcerations and/or scarring to all forms of retinal disease. As shown in the nonlimiting examples below, ESMVs initiated the regeneration of damaged retina by inducing its endogenous regenerative capacity.

The therapeutic ESMV fraction is obtained from native or cultured mammalian ESCs, such as human ESCs, by differential centrifugation, as described in the examples below. Its presence can be confirmed by the presence of known ESC-specific mRNAs (Oct4, Sox2, Nanog, Lin28, Klf4) and microRNAs (miR-292, -294, and -295), as well as by certain ESC-specific surface protein antigens (CD9, Delta 1, integrin a6, integrin 31, sonic hedgehog, sonic hedgehog homolog, SSEA1, SSEA3, SSEA4, and TRA-1-60). Thus, the presence of the therapeutic ESMV fraction can be verified using a certain combination of pluripotency factors. This ESMV fraction may be additionally fractionated to obtain an RNA fraction containing total RNA or certain mRNAs and miRNAs from the ESMVs which can cause de-differentiation of cells or can initiate the expression of genes involved in the development and functioning of certain differentiated ocular cells.

The ESMV fraction may be administered to the eye by any mode of delivery determined to be effective by an ophthalmologist for the disease being treated. For example, intravitreal applications may be appropriate for glaucoma, while AMD and photoreceptor/RPE degenerations may be treated by subretinal injections. Delivery may be bolus, intermittent, or continuous, and may be provided by a device, such as, but not limited to, a delivery pump or contact lens.

In other nonlimiting examples, ESMVs may be administered topically to the eye to treat, e.g., corneal epithelial abnormalities and dry eye. To be administered topically, the therapeutic ESMV fraction may be embedded into an extended release vehicle, such as a hydrogel matrix (see, e.g., Zarembinski, et al. (2011) in Regenerative Medicine and Tissue Engineering-Cells and Biomaterials, Editor: Daniel Eberly, Chapter 16, pp. 341-364), which can be adhered to the side of a contact lens adjacent to the cornea to treat cornea diseases and to stimulate corneal regeneration. The fraction may also be administered continuously or intermittently via a device equipped with a pump.

Diseases of the eye that can be treated according to the disclosure include cell loss within all eye compartments, tear-producing glands, and cells on or near the ocular surface, corneal cell loss, anterior chamber diseases, and the majority of retinal degenerative diseases including, but not limited to, age-related macular degeneration, diabetic retinopathy, glaucoma, the retinitis pigmentosa complex, as well as inherited retinal degenerations. Other diseases of the eye that can be treated according to the disclosure include systemic and local autoimmune disorders that adversely affect the eye, such as, but not limited to, uveitis, dry eye, ocular cicatricial pemphigoid, benign and malignant Mooren's corneal ulcer, and rheumatoid arthritis.

A particular eye disorder may be treated once or multiple times by repeated administrations of the ESMV fraction to the issue affected, as determined by an ophthalmologist. The ESMV fraction may be administered alone or with other therapeutics know to treat the disorder, as long as the secondary treatment does not inactivate the ESMVs being administered.

Reference will now be made to specific examples illustrating the invention. It is to be understood that the examples are provided to illustrate embodiments and that no limitation to the scope of the invention is intended thereby.

### EXAMPLES

### Example 1

### Isolation and Characterization of Mouse ESMVs

Embryonic stem cells (ESCs) derived from the mouse strain SV129 were expanded under serum-free and feeder-free conditions in ESGRO Complete PLUS clonal grade medium supplemented with GSK3β inhibitor to suppress differentiation (Millipore, Billerica, Mass.). 3.5×10⁶ cells were plated on gelatin-coated T175 cm² culture flasks. ESCs were cultured in a humidified 37° C., 5% CO₂ incubator. The growth of ESCs was monitored microscopically and fresh culture medium was added daily and collected every 48 hr for ESMV isolation. ESCs were passaged using ESGRO Complete Accutase (Millipore) every 48 to 72 hr to maintain ESC colonies at 80% confluence in order to maximize ESMV yield while avoiding differentiation of ESCs.

ESC colonies were visually inspected by microscopy on a daily basis for signs of differentiation and Oct4, Sox2, and Nanog mRNA expression was assayed by qRT-PCR using mouse specific primer pairs designed by PrimerQuest^{SM} (Integrated DNA Technology-DNAsite, San Diego, Calif.):

(SEQ ID NO: 1)
Oct4: forward-GCCGGGCTGGGTGGATTCTC,
(SEQ ID NO: 2)
reverse-ATTGGGGCGGTCGGCACAGG,
(SEQ ID NO: 3)
Nanog: forward-TCCAGAA-GAGGGCGTCAGAT,
(SEQ ID NO: 4)
reverse-CTTTGGTCCCAGCATT-CAGG,
(SEQ ID NO: 5)
Sox2: forward-AACAATCGCGGCGGCCCGAGGAG,
(SEQ ID NO: 6)
reverse-GCCTCGGCGTGCCGGCCCTGCG.

To isolate ESMVs, the supernatant was collected in 50 ml centrifuge tubes and spun at 3,500 g for 1 hr at 4° C. to pellet debris and fragmented cells. The supernatant was carefully transferred to an ultracentrifuge tube and spun at 200,000 g for 3.5 hr in a Beckman Type 50.2Ti rotor at 4° C. to pellet the ESMVs. The ESMVs can then be used or fractionated to obtain a therapeutic composition comprising RNA and/or proteins obtained from the vesicles.

### Example 2

### ESMV Fractionation to Isolate RNA

Total RNA was isolated from mouse ESMVs using the mirVana miRNA isolation kit, which retains small RNA species (Ambion, Austin, Tex.), treated with TURBO DNAse (Ambion) to remove DNA traces, and examined by RT-PCR for the presence of mouse Oct4, Sox2, Nanog (primer pairs in Example 1), and Klf4, Lin28, and mmu-mIR-292-3p, -294, and -295 (Taqman® primers) transcripts.

Similarly, RNA in human ESMVs (hESMVs) from hESCs is extracted using the miRNeasy Mini™ kit (Qiagen, Germantown, Md., USA), which isolates total RNA as well as miRNAs. Total RNA is hybridized to Affimetrix GeneChip U133 Plus 2.0 human gene expression arrays (Affimetrix, Santa Clara, Calif.). The target preparations and array hybridizations are performed following the standard Affymetrix GeneChip Expression Analysis protocol. The arrays are scanned using the Affymetrix 7G scanner and the images are acquired using the Affymetrix GeneChip Command Console 1.1 (AGCC). Expressed genes are identified by Affymetrix present calls and are analyzed using Partek genomics Suite 6.4 and RMA algorithm for data normalization. Thresholds for selecting significant genes are set at >=2-fold and an FDR-corrected p<0.05. For microRNA, the Exiqon miRCURY LNA microRNA arrays are used following the manufacturer's instructions (Exiqon, Woburn, Mass. 01801). The miRNA arrays are scanned using the Axon GenePix 4100A scanner and processed with the GenePix Pro 6.0 software. The raw miRNA data are normalized using a combination of housing keeping miRNAs and invariant miRNAs and NLYZED using Partek genomic suite 6.4 with thresholds of >=2-fold and FDR corrected p<0.05. hESMV proteins are characterized by hybridization to Invitrogen ProtoArray Human Protein Microarrays (Invitrogen, Carlsbad, Calif.). hESMV surface antigens are analyzed by flow cytometry for the ESC surface markers 1, integrin α6, integrin β1, sonic hedgehog, sonic hedgehog homolog, SSEA1, SSEA3, SSEA4, TRA-1-60 and TRA-1-81. Multiple batches of hESMVs are isolated from hESC cultures, washed with PBS, resuspended in PBS supplemented with BSA and sodium azide and stained using the corresponding fluorochrome-conjugated monoclonal antibodies. hESMVs are resuspended in the culture medium and taken for flow cytometry analysis. Flow cytometric analysis and the optimization of the experimental conditions are optimized as desired. The acquired data are analyzed using CELLQuest software.

hESMV preparations are screened to ensure absence of bacterial endotoxin in hESMVs using the GenScript ToxinSensor™ Chromogenic LAL Endotoxin Assay Kit, which utilizes a modified Limulus Amebocyte Lysate and a synthetic color producing substrate to quantitatively detect endotoxin chromogenically in a broad range (0.005-1 EU/ml).

### Example 3

### Müller Cell Culture

The human Moorfield/Institute of Ophthalmology-Müller 1 (MIO-M1) cell line, initially derived from postmortem human neural retina, was established and characterized previously (Limb et al. (2002) Invest. Ophthalmol. Vis. Sci. 43:864-869). MIO-M1 cells were maintained as an adherent cell line in 175 cm² tissue culture flasks for propagation, and in 6-well cell culture plates for ESMV treatment experiments, in DMEM medium containing 4500 mg/L glucose, sodium pyruvate and stabilized L-glutamine (GlutaMAX; Invitrogen, Grand Island, N.Y.) with 10% vol/vol fetal bovine serum (filtered, heat inactivated; Gemini Bioproducts, Sacramento, Calif.) and penicillin/streptomycin (Invitrogen) in a humidified 37° C., 5% CO₂ incubator. Upon reaching confluence, the cells were washed with phosphate-buffered saline (PBS), detached from the flasks with trypsin (Invitrogen), washed with complete cell culture medium, and split into fresh flasks. ESMV treatment experiments were started when Müller cells reached 60% confluence.

### Example 4

### ESMV-Induced Morphological Changes in Müller Cells

Müller cells were plated on two 6-well cell culture plates at 1×10⁶ cells per well and allowed to reach 60% confluence prior to initiating ESMV treatments. For these, ESMVs pelleted by ultracentrifugation of media from 6 T175 cm² flasks of mouse ESCs grown in serum free, feeder free conditions (see above), were immediately resuspended in Müller cell medium and equal volume was added to each well of one of the 6-well plates with cultured Müller cells. This procedure was repeated every 48 hr for 9 consecutive treatments. Control Müller cells cultures in the other 6-well plate were subjected only to medium changes in place of ESMV treatments. To maintain 60% confluence, both treated and control cells were passaged as needed at the end of an ESMV treatment. ESMV-exposed and control Müller cells were examined after each treatment using the Leica DM IL LED microscope.

To evaluate the morphological changes induced by ESMVs at the completion of each treatment, Müller cells were fixed in 100% ethanol for 15 min and stained with Harris Hematoxylin and Eosin Y, dehydrated with serial ethanol washes, air dried and coverslipped with ProLong Gold antifade reagent. The transmitted light differential interference contrast images were acquired using the Zeiss Axiovert 135M microscope with a Photometrics CoolSnap camera. To compare the number of cells present in ESMV-exposed and control Müller cell cultures at the end of each treatment, images of 3 to 4 fields of view (acquired at 20× magnification for each well of the 6-well cell culture plates) were obtained using the Leica DCF295 digital camera. Cells within each image were individually marked using Adobe Photoshop, counted, and the treated/control cells ratios were calculated.

At the completion of ESMV treatments, the culture medium of ESMV-exposed Müller cells was aspirated; the cells were then washed 3 times with ample PBS to remove any residual ESMVs and collected for RNA isolation and gene expression studies.

Although the treated and non-treated (control) cultures were initiated from the same passage, number of cells, and confluence level of Müller cells, morphological differences became evident between control and ESMV-exposed cells as early as after the first treatment. In contrast to control cells that grew as uniform, spindle-like, adherent cellular sheets characteristic of typical Müller cells cultures (FIG. 1A), as ESMV treatments progressed, the exposed Müller cells increasingly grew as individual heterogeneous cells, demonstrating decreased cell-cell adhesion, presence of cells with multiple processes, stellate cells, multinucleated cells, and cells with unilateral boutons and extensive processes (FIGS. 1B and 1C). Often, the nuclei of ESMV-treated cells were enlarged, many demonstrating visible metaphase plates. Cell count comparison between ESMV-treated and control cultures did not reveal significant decline in the overall number of cells in the treatment group (FIG. 1D).

Alternatively, ESMV-exposed and control Müller cells were examined after each treatment using the Leica DM IL LED microscope (Leica Microsystems, Wetzlar, Germany), and for the determination of cell number, images of 3 to 4 fields of view (acquired at 20× magnification for each well of a 6-well plate of treated and control cells) were obtained using a Leica DCF295 digital camera. Cells within each image were individually marked using Adobe Photoshop (Adobe Systems, San Jose, Calif.), and then counted. Cell number per field of view was obtained at each time point for treatment and control groups and ratios of treated/control cells was calculated. For morphology studies, Müller cells were fixed in 100% ethanol for 15 min and stained with Harris Hematoxylin and Eosin Y (Fisher Scientific, Pittsburgh, Pa.), dehydrated with serial ethanol washes, air dried and coverslipped with ProLong Gold antifade reagent (Invitrogen). The transmitted light differential interference contrast images were acquired using a Zeiss Axiovert 135M microscope with a Photometrics CoolSnap camera (Roper Scientific, Tucson, Ariz.).

### Example 5

### Analysis of RNA in ESMV-Treated MüLler Cells

hESMVs are added to human Müller cell cultures by resuspension in Müller cell medium and RNA is isolated at 8 hr, 24 hr, and 48 hr post-treatment from ESMV-treated and untreated cells. For the initial analysis of gene expression changes in Müller cells post-ESMV treatment, total RNA was isolated using the mirVana™ miRNA Isolation Kit (Ambion) from ESMV-treated and from control Müller cells cultured under three different conditions (Müller cells not exposed to ESMVs, Müller cells incubated with ESGRO medium components that remained after ultracentrifugation at 200,000 g for 3.5 hr, and Müller cells treated with the components of the conditioned medium of MEF cultures, after ultracentrifugation at 200,000 g for 3.5 hr). The RNA was quantified and quality assessed using a Nanodrop ND-1000 spectrophotometer (Thermo Scientific, Wilmington, Del.) and treated with TURBO DNAse (Ambion) prior to further manipulation. RNA was converted to cDNA using SuperScript™ III First-Strand Synthesis SuperMix for qRT-PCR (Invitrogen). To analyze embryonic gene transfer from mouse ESMVs to human Müller cells, mouse-specific primer pairs described above for Oct4, Sox2, and Nanog were used, and amplification was detected using Brilliant Sybr Green qPCR Master Mix (Stratagene, La Jolla, Calif.) in an Mx3000p qPCR instrument (Stratagene). All results were normalized to the human housekeeping gene glyceraldehyde 3-phosphate dehydrogenase (Gapdh), amplified using commercially available primers (IDT, Coralville, Iowa). The relative change in gene expression was determined using the 2^{-ΔΔCt} method of comparative quantification.

To detect the induction of expression by ESMVs of endogenous embryonic and early retinal genes in human Müller cells, TaqMan® primers for the human Oct4, Pax6, and Rax genes and the TaqMan® Gene Expression Assays protocol and reagents (Applied Biosystems, Carlsbad, Calif.) were used; TaqMan® Gapdh primers were used for normalization.

With the use of qRT-PCR and species-specific primers, the transfer by ESMVs of mRNA transcripts from mouse ESCs was distinguished from the induction by ESMVs of endogenous transcripts of human Müller cells. While mouse Oct4 and Sox2 mRNAs were transferred from ESMVs and remained elevated in Müller cells 48 hr post-ESMV treatment (FIGS. 2A and 2B), no Nanog transfer was observed at any time point post-treatment (FIG. 2C), indicating that ESMVs transfer genetic information by a selective mechanism.

Human Oct4 mRNA in ESMV-treated Müller cells was increased 3-fold as early as 8 hr post-ESMV exposure and remained elevated for the next 40 hr (FIG. 2D), indicating that the induction of the endogenous Oct4 mRNA of Müller cells by ESMVs begins shortly after exposure and persists for days. The levels of Pax6 and Rax mRNAs, which encode transcription factors expressed throughout retinogenesis by multipotent retinal progenitor cells were found elevated 8 hr post-ESMV exposure and persisted at the 48 hour time point (FIGS. 2E and 2F).

That the results above were specific to the ESMV treatment was corroborated by using two other controls in addition to the Müller cells that had not been exposed to ESMVs: (a) the residue from equal volume of ESGRO Complete PLUS medium as that used in the isolation of ESMVs from ESCs, after ultracentrifugation at 200,000 g for 3.5 hr, was re-suspended in Müller cell culture medium and then incubated with Müller cell cultures for 8 hr, 24 hr, or 48 hr to determine whether it can modify the expression of the specific mRNAs; and (b) the same volume of medium from a culture of mouse embryonic fibroblasts (MEFs) as that used in the isolation of ESMVs from ESCs was processed as in (a) to see if microvesicles released by cells different from ESCs can also change the expression of the studied mRNAs.

After qRT-PCR with the specific primers, the levels for mouse Oct4 and Sox2 as well as human Oct4, Pax6 and Rax mRNAs were the same in Müller cells not exposed to ESMVs and in control Müller cells treated with the ESGRO medium components or with the MEF conditioned medium. These results demonstrate that only incubation with ESMVs changes the specific mRNA levels in Müller cells.

miRNAs, small noncoding RNAs, are important regulators of gene expression and maintenance of ESC pluripotency and cell fate determination. ESMVs are highly enriched in miRNAs [6], including ESC-specific miRNAs of the 290-cluster, which are involved in maintenance of ESC pluripotency.

### Example 6

### miRNA Analysis

After obtaining total RNA from ESMV-treated and control Müller cells, comparative quantification of mmu-mIR-292-3p and mmu-mIR-295 with snRNA U6 (endogenous control) was performed in 3 to 6 biological samples, ran in parallel, using TaqMan® miRNA qRT-PCR assays and TaqMan® probes (Applied Biosystems) according to manufacturer's protocol, each primer ran in triplicate, and the 2^{ΔΔCt} method was used to examine the fold-change of miRNA levels. The significance of these changes was assessed using Student's t-tests. The commercially available primer sequences for the TaqMan® assays can be downloaded from the manufacturer's website (https://bioinfo.appliedbiosystems.com/genome/database/gene/expression.html).

To demonstrate that the transfer of miRNAs is likely to be one of the mechanisms by which ESMVs influence gene expression in Müller cells, the following experiment was performed. Using qRT-PCR, the presence of miRNA-292 and -295 was tested for in Müller cells at 8 hr, 24 hr, and 48 hr post-ESMV treatment, using U6 snRNA, a small nuclear RNA ubiquitous in mammalian cells, as normalizer. Since mature miRNA transcripts are very short, a strategy that uses a stem-loop RT primer was used. To avoid confounding levels of such small transcripts by enrichment methods for ESMVs, the same total RNA samples used earlier for mRNA transfer studies to assay miRNA transfer were used.

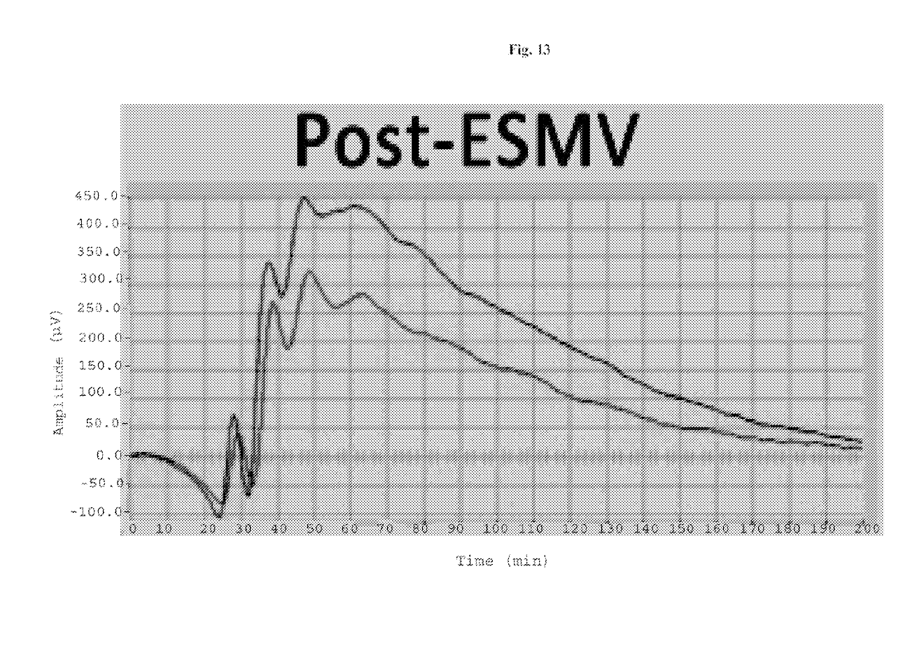

Both miRNA -292 (FIG. 3A) and 295 (FIG. 3B) were found to transfer efficiently to Müller cells and persist for 48 hr post-treatment, indicating that miRNAs are not degraded and play a role in gene expression alterations of Müller cells.

In other studies, ESMVs are added to human Müller cell cultures by resuspension in Müller cell medium and RNA is isolated at 8 hr, 24 hr, and 48 hr post-exposure from ESMV-treated and untreated cells.

RNA expression changes in ESMV treated versus untreated Müller cells are identified by hybridization to Affimetrix GeneChip U133 Plus 2.0 human gene expression arrays (Affimetrix, Santa Clara, Calif., USA), as described above.

mRNAs important for maintenance of stem cell pluripotency, Oct4, Sox2 and Nanog, stem cell specific miRNAs 292 and 295, and early retinal transcripts Pax6 and Rax are examined by qRT-PCR, using Taqman™ Assays according to the manufacturer's protocol, as described above.

### Example 7

### Analysis of Gene Expression in ESMV-Treated Retinal Progenitor Cells

The transcriptional response of Müller cells after 8 hr, 24 hr, and 48 hr of ESMV exposure was compared with the transcriptome of control Müller cells cultured for the same number of hours by hybridization of cDNA from 3 (8 hr and 24 hr) and 2 (48 hr) independent biological samples of control and ESMV-treated Müller cells, each in triplicate, to Agilent human 8×60K cDNA arrays (Agilent, Santa Clara, Calif., USA). The RMA algorithm was used for data normalization [30]. The minimum thresholds for selecting significant genes were set at ≧3 log₂-transformed fold-change and FDR-corrected p<0.001. Genes that met both criteria simultaneously were considered significantly changed.

Known marker genes of Müller glia were detected in the microarrays (glutamine synthetase (Glu1), clusterin (Clu), dickkopf homolog 3 (Dkk3), aquaporin 4 (Aqp4), S100 calcium binding protein A16, Apolipoprotein E (ApoE), Vimentin (VIM), and glial fibrillary acidic protein (GFAP)).

1894 genes were differentially expressed at all 3 time points post-ESMV treatment, with 801 genes up- and 1093 genes down-regulated (FIG. 4A). Tight clustering of genes in ESMV-treated versus control Müller cells was observed, with treated cells sharing a similar gene expression profile over a wide range of genes (FIG. 4B). More than 60% of the gene expression changes occurred by 8 hr post-treatment. 1444 genes were up- and 1878 genes were down-regulated at 8 hr, 1623 genes were up- and 1828 genes were down-regulated at 24 hr, and 1711 genes were up- and 1907 genes were down-regulated at 48 hr post-ESMV treatment. The majority of gene expression changes (95%) occurred by 24 hr, with only 624 genes unique to the 48-hour time point (FIG. 4A).

Gene ontology (GO) analysis revealed that many of the genes differentially regulated at 24 hr and 48 hr post-treatment belonged to the transcription factor families, genes involved in retinogenesis, organ and organismal development, and genes encoding cell-cell signaling molecules, receptors involved in morphogenesis, multiple cytokines and immune response genes. Grouped by functional category (FIG. 5A), ESMV-treated Müller cells differentially expressed multiple genes involved in cellular movement and extracellular matrix composition, inflammation, cellular growth and proliferation, tissue response to injury, molecular transport, energy metabolism, embryonic development, cell survival, DNA replication, and genes involved in ophthalmic diseases (Table S1). Many of the 1894 genes differentially expressed in ESMV-treated Müller cells at all time points have been linked, among others, to the following canonical signaling pathways: communication between innate and adaptive immune cells, G-protein coupled receptor signaling, vitamin D receptor and retinoic acid X receptor activation, (involved in the development of neural retina), IL6 signaling (retino-protective pathway), neuregulin signaling (a pathway which plays a role in promoting retinal neuron survival and neurite outgrowth in developing retina (Bermingham-McDonogh et al. (1996) Development 122:1427-1438)), and axonal guidance (FIG. 5B).

Among the up-regulated genes were pluripotency genes Oct4, Lin28, Klf4, and LIF, early retinal genes Bmp7, Olig2, FoxN4, Dll1, Pax6, and Rax, genes IL6, CSF2 with known retinal protective properties, and inducers of retinal regeneration (FGF2, IGF2, GDNF), as well as multiple extracellular matrix modifying molecules, such as the gene for Matrix metalloproteinase 3 (MMP3), that are known to create permissive environment for tissue remodeling. Among the down-regulated genes were those promoting differentiation, such as DNMT3a and GATA4, inhibitory extracellular matrix components, such as Aggrecan, heparin sulfate, and Tenascin, and inhibitory scar tissue components, such as GFAP and chondroitin sulfate proteoglycans. Expression changes in these genes were more pronounced at 24 hr and 48 hr post-treatment. While the expression of c-Myc, a well-characterized pluripotency-inducing factor, was detected in Müller cells, it remained unchanged throughout the course of ESMV treatments. Hes1, Notch 1, Notch2, and NeuroD1, genes which regulate cell cycle re-entry, de-differentiation, and activation of retinal stem cell phenotype in Müller cells, were highly up-regulated at 8 hr post-ESMV treatment, with levels remaining increased over baseline, but declining at other time points. The expression of EGFR, a gene involved in driving retinal progenitors towards Müller glial fate during retinogenesis was down-regulated at all three time points.

The observed changes in the transcriptome of these Müller cells induced by ESMV treatments shows a shift towards a more de-differentiated state, possibly through the activation of the proliferative and regenerative programs of these cells.

Additionally, microarray data analysis revealed the up-regulation of several genes encoding markers of various retinal lineages in Müller cells exposed to ESMVs, including those for calbindin 1, a marker of horizontal and amacrine retinal neurons, Syntaxin 1a, a marker of amacrine cells, and rhodopsin, a marker of rod photoreceptors. The expression of calbindin 1 was highest 48 hr post-ESMV, while the expression of rhodopsin and Syntaxin 1a was increased at all tested time points.

These findings demonstrate that subsets of de-differentiating Müller cells trans-differentiate into cells of other retinal lineages.

### Example 8

### Analysis of miRNA Expression in ESMV-Treated Retinal Progenitor Cells

miRNAs play a role in retinogenesis, regulating retinal progenitor cell progression from early to late stages and their differentiation towards various retinal cell lineages. Accordingly, testing was done to determine if miRNAs delivered to Müller cells by ESMVs alter the miRNA and mRNA expression profiles of Müller cells and shift these cells towards a de-differentiated state.

One μg total RNA samples prepared as described above was labeled with Hy3™ and the labeled miRNAs were hybridized to miRNA arrays. Exiqon miRCURY LNA miRNA arrays (microarrays v11), which include 927/648/351 human/mouse/rat miRNAs as well as 438 miRPlus miRNAs, were used according to the manufacturer's instructions. The miRNA array slides were scanned with an Axon GenePix 4100A scanner (Molecular Devices, Sunnyvale, Calif.) and processed with the GenePix Pro 6.0 software (Molecular Devices). The raw miRNA data were normalized using a combination of housekeeping miRNAs and invariant miRNAs. Statistically different miRNAs were selected using Partek genomic suite 6.4 with thresholds of ≧3-fold and FDR corrected p<0.05. Individual miRNAs were studied using the miRBase online database (http://www.mirbase.org/), and miRNA target prediction analysis was performed using TargetScan 6.0 software (http://www.targetscan.org/).

### Results

Overall, the expression of 173 miRNAs was altered by ESMV treatment; 25 of these miRNAs were differentially expressed at all three time points tested (FIG. 7A), with 11 up- and 14 down-regulated (FIG. 7A). 25 miRNAs were up- and 16 down-regulated at 8 hr, 32 miRNAs were up- and 28 down-regulated at 24 hr, and 87 miRNAs were up- and 61 were down-regulated at 48 hr post-ESMV treatment (FIG. 7B). The majority of alterations in miRNA expression occurred by 48 hr post-ESMV exposure.

Several of the miRNAs which are highly expressed in developing retina were up-regulated in ESMV-treated Müller cells, including miR-1, miR-96, miR-182 and miR-183. miRNAs belonging to the 290 cluster (miR-291b-5p, -292, -294, and -295), the miRNA cluster involved in the maintenance of ESC pluripotency, were up-regulated and remained increased over 48 hr post-ESMV exposure. Concurrently, the expression of miR-let-7b and miR-let-7c, belonging to the miR-let-7 cluster which inhibits cell cycle progression and promote cell differentiation, decreased post-ESMV treatment. miR-7, which represses the expression of Yan protein and promotes photoreceptor differentiation [44], as well as miR-125-2b, highly abundant in adult retina, were down-regulated over 48 hr post-ESMV treatment. Among the miRNAs strongly up-regulated at all three time points tested were miR-133a (increased 30-fold) and miR-146a (increased 37-fold), the miRNAs which promote cell proliferation and inhibit differentiation of skeletal myoblasts and myogenic stem cells, respectively, the latter acting via the Notch signaling pathway, the same pathway which regulates retinal progenitor differentiation. Among the miRNAs strongly down-regulated at all tested times were miR-199b-5p (decreased 70-fold), miR-214 (decreased 37-fold), and miR-143 (decreased 13-fold), which promotes differentiation of ESCs, neuroblasts, and smooth muscle progenitors, respectively (Cordes et al. (2009) Nature 460:705-U780; Letzen et al. (2010) PLoS One 5:e10480; Fischer et al. (2001) Nat. Neurosci. 4:247-252) (FIG. 8). The observed profile of miRNA expression changes in Müller cells post-ESMV exposure demonstrates de-differentiation, consistent with that observed for mRNA expression changes.

Several miRNAs involved in maintenance of pluripotency (miR-294, -146a, -133a) and differentiation (miR-199b-5p, -214, -143) were selected for validation of the microarray results. Total RNA samples from Müller cells at 24 hr and 48 hr post-ESMV treatment (the time points corresponding to the majority of miRNA expression changes) and from untreated control cells were subjected to qRT-PCR using TaqMan miRNA Assays that included stem-loop RT primers specific for each miRNA. qRT-PCR results confirmed the pattern of expression observed by the microarray screening for all the miRNAs tested (FIG. 8).

### Example 9

### Immunocytochemical Analysis of ESMV-Induced Retinal Progenitor Cell Transdifferentiation

To further characterize the morphologically heterogeneous cell population observed in the cultures of Müller cells treated with ESMVs and validate the microarray data, the expression of markers of various retinal cell lineages in ESMV-treated Müller cells was investigated compared to untreated control cells by immunocytochemistry. Results

Müller cells that had had 8 treatments with ESMVs were seeded on poly-D-lysine-coated glass coverslips placed in the 6-well culture plates, allowed to attach, and treated with ESMVs derived from 6 T175 flasks, as described above. 24 hr later, ESMV-treated and control cells were rinsed in 0.1 M PBS and fixed for 30 min in 4% paraformaldehyde (Electron Microscopy Sciences, Hatfield, Pa.), then rinsed in 0.1 M PBS and blocked in 10% serum containing 1% bovine serum albumin (BSA) and 0.5% Triton X-100 for 1 hr at RT. Primary and secondary antibodies were diluted with PBS containing 3% serum, 1% BSA and 0.5% Triton X-100. Cells were incubated with the following primary antibodies, overnight, at 4° C.: rabbit monoclonal anti-Brn3a (1:500, Abcam, Cambridge, Mass.), mouse anti-Neuronal Nuclei (NeuN) monoclonal antibody (1:500, Millipore) mouse anti-HPC1 (Syntaxin 1a) monoclonal antibody (1:1000, Sigma, St Louis, Mo.), mouse anti-glutamic acid decarboxylase 67 (Gad67) monoclonal antibody (1:000, Millipore), mouse anti-1D4 (rhodopsin) monoclonal antibody (1:10,000, Millipore), rabbit anti-GS polyclonal antibody (1:1000, Sigma), mouse anti-parvalbumin monoclonal antibody (1:1000, Swant, Marly, Switzerland) and guinea pig anti-vesicular glutamate transporter 2 (vGluT2) (1:10,000, Millipore). ESMV-treated and control cells were then incubated for 1-2 hr at RT with the appropriate secondary antibodies conjugated to AlexaFluor488, AlexaFluor568, or AlexaFluor594 (Molecular Probes, Eugene, Oreg.) and diluted 1:1000. Coverslips were mounted on slides using ProLong® Gold Antifade Reagent containing the nuclear counterstain DAPI (4,6-diamidino-2-phenylindole; Invitrogen), allowed to dry, and images were obtained with an Olympus FluoView FV 1000 confocal laser scanning microscope using Olympus FluoView software for capture and processing (Olympus America, Center Valley, Pa.).

In addition to the Müller cell marker, glutamine synthetase (GS) (FIGS. 9A and 9B), immunoreactivity to Gad67, a marker of amacrine and horizontal cells (FIGS. 9A and 9C), NeuN, a marker of amacrine and retinal ganglion cells (FIGS. 9G and 9I), Bm3a, a marker of retinal ganglion cells (FIG. 9M), and Syntaxin 1a, a marker of amacrine cells (FIG. 9O) were observed in small populations of ESMV-treated Müller cells. None of these markers were present in the untreated control cultures (FIGS. 9G-9L, 9N, 9P, and 9R). Interestingly, immunoreactivity to rhodopsin, a marker of rod photoreceptors, was seen primarily localized in cytoplasmic granules in a very small number of treated cells (FIG. 9Q). No immunoreactivity to Parvalbumin, a bipolar and horizontal cell marker, or Vesicular Glutamate Transporter 1, a marker for bipolar and photoreceptor cell terminals, was found. No staining was observed when primary antibodies were omitted.

This data suggest that ESMV treatment induces transdifferentiation of Müller cells into cells of retinal neural lineage, mainly towards amacrine and retinal ganglion cells, but not horizontal or bipolar cells. The very limited expression of rhodopsin post-ESMV exposure also suggests that ESMV treatment induces at least a partial activation of genes of photoreceptor lineage.

### Example 10

### Validation of Transcript Level Changes in Treated Retinal Progenitor Cells

As verified with qRT-PCR of RNA from Müller cells (which are representative of the regenerative cell population) subjected to ESMV treatment for 24 hr and 48 hr the expression profiles of a subset of genes involved in the processes of de-differentiation (Cyclin D2, BMP7), retinal protection (IL6, IGF2), repair and tissue remodeling (MMP3), as well as genes involved in scar formation (GFAP) and inhibition of ECM components (Aggrecan), all of which were significantly altered in the ESMV-treated group on the microarrays. These time points were chosen because the majority of changes in the expression of genes involved in retinal protective and regenerative processes were observed within 24 hr and 48 hr.

All tested genes demonstrated the same pattern of regulation as observed in microarrays (FIG. 6). In particular MMP3 mRNA, which encodes a matrix metalloproteinase that up-regulates in newt adult organ repair, including retina, and facilitates the integration into the retina of transplanted photoreceptors when present at elevated levels, was found strongly up-regulated in microarrays and qRT-PCR experiments, as was IL6 mRNA; its expressed interleukin has been demonstrated to have a protective effect on inner retina neurons. On the other hand, the Aggrecan gene, which encodes a chondroitin sulfate proteoglycan required for normal glial cell differentiation and development was among the genes down-regulated in microarrays and qRT-PCR studies, as was GFAP, a gene that when deleted from the mouse genome improves retinal transplant integration.

qRT-PCR analysis of independent samples of ESMV-treated and control Müller cells was carried out to validate the expression changes from the mRNA and miRNA microarray data. Since microarray analysis indicated that the majority of expression changes in the genes of interest take place 24 hr and 48 hr post-ESMV exposure, these time points were used for array validation. Total RNA was isolated using miRNeasy Mini kit (Qiagen) and subjected to on-column DNase digestion per protocol. For gene expression change validation, RNA was converted to cDNA as described above, and qPCR was carried out using the following TaqMan® primers, selected to span exon-exon junctions to eliminate potential genomic DNA amplification in the Expression Assay protocol (Applied Biosystems, https://products.appliedbiosystems.com/ab/en/US/adirect/ab?cmd=catNavi-gat2&catID=601803): BMP7, IL6, MMP3, IGF2, Cyclin D2, Aggrecan, and GFAP, with Gapdh serving as the endogenous control. In each experiment, a sample without reverse transcrip-tase and a sample without template were included to demonstrate specificity and lack of DNA contamination.

For miRNA array validation the following TaqMan® miRNA assays (Applied Biosystems, link above) were used in accordance with manufacturer's protocol: hsa-mIR-146a, hsa-miR133a, mmu-mIR-294, hsa-mIR-199-5p, hsa-mIR-214*, hsa-mIR-143, normalized against snRNA U6. Fold-change in gene expression was calculated using the 2^{-ΔΔCt} method for each mRNA and miRNA tested; 3 biological replicates were ran in parallel for each sample and each primer was ran in triplicate. Student's t-test was used to assess significance of gene expression change.

### Example 11

### Verification of Therapeutic Activity of hESMVs in NMDA-Damaged Mouse Retinas

The results from the above studies on exposing retinal glial and Müller cells, to ESMVs suggest that in retina, ESMVs induce de-differentiation of quiescent glial progenitor Müller cells to a stem cell phenotype, cell cycle re-entry, changes in Müller cells' microenvironment towards a more permissive state for tissue regeneration and a retinogenic program leading to regeneration.

In initial studies on 27 mice, damage to retinal ganglion cells (RGCs) and inner retinal neurons of young adult (about 7 weeks old) animals was induced by intravitreal injection of N-Methyl-D-Aspartic acid (NMDA, an excitotoxin which, by activating the NMDA receptor, causes an intracellular influx of calcium ions, generation of reactive oxygen species, and ultimately leads to the apoptosis of neural cells in both the ganglion cell and inner nuclear layers of the retina) into both eyes of each mouse. Cell death of the affected neurons occurs within 24 hours of NMDA administration; thus, 48 hours later, 1 μl of ESMV suspension (50 μg to 250 g) containing BrdU and fluorescein was injected subretinally into the left eyes while the right damaged eyes (controls) were injected with 1 μl of phosphate buffered saline (PBS) containing the same concentrations of BrdU and fluorescein than the ESMV suspension. (Injections can also be done intravitreally with or without subretinal injections.) Because most intravitreal BrdU is cleared from the eye within 6 hours, BrdU was injected intraperitoneally on the day of injection and daily thereafter for the next 6 days. Cells that proliferate in the NDMA-damaged retinas post-ESMV exposure express CRALBP (a Müller glial marker), Syntaxin 1A, and Gad67 (both amacrine cell markers).

### Results

While no BrdU-labeled cells were detected by fluorescent microscopy in the PBS-injected retinas, BrdU staining of a small number of cells was observed in the ESMV-treated eyes at 7 days post-injection (FIG. 10B, FIG. 11B, and FIG. 12B), as well as 30 days post-injection (FIG. 10E, FIG. 11E, FIG. 12E). BrdU-positive cells were located in the innermost rows of the inner nuclear layer (INL), a region occupied primarily by nuclei of amacrine neurons and in the GCL (FIG. 10B, FIG. 11B, and FIG. 12B). At 30 days post-ESMV injections (FIG. 10E, FIG. 11E, and FIG. 12E), the number of BrdU positive cells had decreased from that at 3 days post-injection (FIG. 10B, FIG. 11B, and FIG. 12B), but BrdU positive cells were still found in the inner plexiform layer (IPL), proximal to the INL. The majority of proliferating cells expressed CRALBP, a well-characterized Müller cell marker (FIG. 12A and FIG. 12D), demonstrating that Müller cells proliferate in response to the ESMV treatment. Some of the proliferating cells examined at 30 days post-ESMV injections expressed Syntaxin 1a (FIG. 11D) and Gad67 (FIG. 10D), indicating that they differentiated along the amacrine lineage. Moreover, a striking improvement was observed in the ERG b-wave 30 days post-ESMV injection (amplitude about 51% to 65% higher than after NMDA-damage) in 5 out of 9 animals, reflecting recovery of retinal function by ESMV treatment (FIG. 13).

### Example 12

### Human ESMV Isolation

H1 and H9 human ESC lines (hESCs) are cultured and expanded under serum-free, feeder-free, conditions. Briefly, hESCs are grown on CELLstart™ CTS™ defined substrate (Invitrogen) in a 1:1 ratio of two defined xeno-free media typically used to culture hESC without feeders, TeSR2 and Nutristem, (Invitrogen). Cells are maintained with daily change of medium. In these conditions, the hESCs show typical undifferentiated morphology and express the pluripotency markers Nanog, Oct4 and Sox2. However, to maintain the consistency of hESC composition, the cultures are examined daily, and any colony that appears to have differentiated is manually removed prior to changing the medium. Cells are passaged every 4 to 6 days mechanically (Karumbayaram et al. (2012) Stem Cells Transl. Med. 1 (1):36-43). A confluent plate is usually split 1:6.

For hESMV collection, the media from day 4 to 6 cultures of H1 and H9 hESCs is collected and spun at 3,500 g for 1 hr to pellet debris and fragmented cells. The supernatants then undergo serial ultracentrifugations at 200,000 g and washing steps to obtain the purified hESMV pellets (Katsman et al. (2012) PLoS ONE 7 (11):e50417)). Protein and RNA content in the ESMV preparations is measured to corroborate the consistency of the isolation protocol.

hESMVs are tested for mycoplasma, endotoxin, aerobic and anaerobic bacteria and fungi by the UCLA Clinical Microbiology laboratory.

### Example 13

### hESMV Characterization

The mRNAs, microRNAs, and proteins of hESMVs derived from the two hESC lines described above are compared with mESMVs, since these cells show differences in their responsiveness to extrinsic signals and in the expression of various markers (Ginis et al. (2012) Dev. Biol. 269(2):360-380). RNA in hESMVs from H1 and H9 hESCs and in mESMVs from a mESC line that generated according to the protocol described in Yuan et al. ((2009) PLoS One 4 (3):e4722) is extracted using the miRNeasy Mini™ kit (Qiagen), which isolates total RNA as well as miRNAs. Each RNA sample is divided into fractions. One fraction is used for mRNA and the other for miRNA analysis. The presence of ESC-specific mRNAs (Oct4, Nanog, Sox2, Lin28, and Klf4) and miRNAs of the 290 and 302-367 clusters is analyzed by qRT-PCR using the relative standard curve method of RNA quantification. In addition, human and mouse total RNAs are hybridized to the Affimetrix GeneChip U133 Plus 2.0 human gene expression array and GeneChip Mouse Genome 430 2.0 expression array, respectively, by the UCLA Clinical Microarray Core facility, following the standard Affymetrix GeneChip Expression Analysis protocol. The arrays are scanned using the Affymetrix 7G scanner and the images are acquired using the Affymetrix GeneChip Command Console 1.1. Expressed genes are identified by Affymetrix present calls. Human and mouse differentially expressed genes are analyzed using Partek genomics Suite 6.4, and the RMA algorithm is used for data normalization. Thresholds for selecting significant genes are set at >=2-fold and an FDR-corrected p<0.05. Genes that meet both criteria simultaneously are considered as significantly different.

Human and mouse microRNA analyses also are performed. The Exiqon miRCURY LNA microRNA array is used following the manufacturer's instructions. The miRNA arrays are scanned using the Axon GenePix 4100A scanner and processed with the GenePix Pro 6.0 software. The raw miRNA data is normalized using a combination of housekeeping miRNAs and invariant miRNAs. Statistically different miRNAs are selected using Partek genomic suite 6.4 with thresholds of >=2-fold and FDR corrected p<0.05.

Transcript and miRNA level differences between human and mouse ESMVs found with the microarray experiments are validated by qRT-PCR analysis of the corresponding mRNAs and miRNAs in hESMV and mESMV samples. Total RNA is converted to cDNA using the SuperScript™ III First-Strand Synthesis SuperMix for qRT-PCR (Invitrogen) and qPCR are carried out using TaqMan® primers, selected to span exon-exon junctions to eliminate potential genomic DNA amplification (Applied Biosystems), with Gapdh for cDNAs or snRNA U6 for microRNAs serving as the endogenous controls. Fold difference in gene expression is calculated using the 2^{-ΔΔCt} method for each gene and miRNA tested, 3 biological replicates are run in parallel for each sample. Student's t-test is used to assess significance of expression differences.

hESMV proteins are characterized by hybridization to an Invitrogen ProtoArray Human Protein Microarray. The presence and quantity of proteins found to stimulate regeneration of damaged retinas using mESMVs, such as IL6, FGF2, and IGF2, are examined in hESMVs by Western blot analysis.

hESMV and mESMV surface antigens are compared by flow cytometry for the ESC surface markers CD9, CD133, Delta 1, integrins α6 and β1, sonic hedgehog, Thy1, SSEA1, SSEA3, SSEA4, TRA-1-60, and TRA-1-81. Multiple batches of hESMVs and mESMVs are isolated from hESC and mESC cultures, respectively, resuspended in PBS supplemented with BSA and sodium azide and stained using the corresponding fluorochrome-conjugated monoclonal antibodies. Optimization of the experimental conditions, as needed, and flow cytometry analysis is carried out at the UCLA's Flow Cytometry Core Laboratory. The acquired data is analyzed using CELLQuest software.

### Example 14

### Alternative Isolation and Characterization of hESMVs

If some mRNAs or miRNAs are highly expressed in the H1-derived hESMVs and not in the H9-derived ones, while the opposite happen to others, an alternative method of testing is used.

Müller cells are cultured with hESMVs (similar amount of protein as in mESMV incubations) for 8 hr, 24 hr, and 48 hr. The morphological changes are evaluated by comparison with untreated control Müller cells, as done after exposure to mESMV. Following aspiration of the culture medium, Müller cells are washed 3 times with ample PBS to remove any residual hESMVs. RNA is then isolated from the cells and gene expression changes (mRNAs and miRNAs) are determined by RT-qPCR.

### Example 15

### Regeneration of Damaged Retinas Using hiPSMVs

The following testing is performed to determine whether human-induced pluripotent stem cell microvesicles (hiPSMVs), human microvesicles obtained from cultured human-induced pluripotent stem cells (hiPSCs), have a comparable mRNA and miRNA composition to that of the chosen hESMVs, and to test the effect of hiPSMVs on cultured progenitor retinal Müller cells. Several lines of hiPSCs have been generated according to the protocol described in Karumbayaram et al. ((2012) Stem Cells Transl. Med. 1 (1):36-43)). The NHDF and Fibrogro hiPSC lines are used which were derived and reprogrammed (Sommer et al. (2009) Stem Cells 27:543-549) under xeno-free conditions and characterized as per GMP. Microvesicles are isolated from passage 15 hiPSCs following the same protocol used to obtain hESMVs from hESCs described above, and the effects of hiPSMVs on Müller cells are tested as described above for hESMVs.

When the selected hESMVs show in in vitro studies that they produce similar results on progenitor Müller cells to those observed with mESMVs, the corresponding GMP grade hESC line is obtained from WiCELL, Wisconsin. These cells are cultured under GMP compatible conditions at the UCLA iPSC GMP laboratory, along with the in-house GMP compatible hiPSC line chosen, and GMP grade hESMVs as well as hiPSMVs are obtained following the protocol established above for the study of their in vivo effect on mice.

### Example 16

### Regeneration of Damaged Retina by hESMV of the Acutely NMDA-Injured Mouse Model

To determine if ESMVs induce the endogenous regenerative capacity of damaged retina, stimulating the quiescent resident progenitor cells to de-differentiate, proliferate and turn on an early retinogenic program, possibly repopulating the retina, the following testing is done.

1. Retinal Function

Scotopic and photopic ERGs are determined at the Jules Stein Eye Institute LIFE (Life Imaging and Functional Evaluation) Core facility. An advantage of ERGs is that they allow the evaluation of the function of the retina in living animals; ERGs can be repeated consecutively to determine function over time. Recordings are obtained from each eye of a cohort of C57B1/6J mice to collect information about their retinal function prior to the beginning of the study. Every retina is then acutely damaged by intraocular injections of NMDA, and ERGs are performed the next day on both eyes of each mouse to assess the injury level. Mice are divided into two groups. On day 3 post-NMDA injections, one group is injected intraocularly and the other subretinally with a range of doses (10 ng, 25 ng, 50 ng, 175 ng, and 250 ng RNA/μl of sterile saline) of the hESMVs selected as described above. One eye receives the hESMV injection, while the other serves as control and receive a PBS injection. To compare the delivery methods for efficacy, retinal function is examined by ERG in the intraocularly- and subretinally-injected mice at 14 and 30 days post-hESMV injection, and the % of functional recovery is calculated from the peak amplitudes of the ERG b-waves in hESMV-treated and untreated eyes. A subset of animals from each experimental group is sacrificed at the same time points, and their eyes fixed and processed for morphological studies using confocal and electron microscopy. Improvement of retinal morphology is correlated with ERG findings to determine the most effective delivery method and dose of hESMVs, which are used in subsequent studies.

To obtain statistically significant, reproducible therapeutic activity of GMP compatible hESMVs in mice with NMDA-injured retinas, the dose and delivery method found to be the most effective is used to treat the left eye with hESMVs and the right eye with PBS of mice from 3 cohorts, each with 15 animals. Scotopic and photopic ERG responses from the eyes of 8 untreated mice and from both eyes of each mouse from Cohort 1 are recorded 14 and 30 days after hESMV injection. Functional recovery and cellular rescue are quantified. In addition, mice from Cohorts 2 and 3 receive a second dose of hESMVs 5 days after the first injection, and Cohort 3 animals are given a third hESMV dose 5 days later. The ERGs from the eyes of the 30 mice from Cohorts 2 and 3 are also recorded at days 14 and 30 after the first hESMV injection. The results obtained enable the determination of whether repeated hESMV treatments are necessary to ensure a better outcome.

### Example 17

### Characterization of Cells Proliferating Post-hESMV Treatment

Using immuno-histochemistry, the marker of cell proliferation bromodeoxyuridine (BrdU) is co-localized with retinal cell-specific markers. Left eyes from mice with NMDA-damaged retinas are injected with BrdU along with hESMVs as described above. The right eyes of the same mice are used as controls and receive BrdU diluted in PBS. At the appropriate time points, animals are perfused and their eyes enucleated, fixed, and processed for immunohistochemistry. Retinal sections are double- and triple-stained with antibodies to BrdU and retinal cell-specific markers (rhodopsin (rods), cone opsin (cones), Brn3a (ganglion cells), Syntaxin 1a and Gad67 (amacrine cells) and glutamine synthetase (Müller cells)) in order to determine which cell types are responding to hESMVs exposure by proliferating and repopulating the retina.

### Example 18

### Safety Profile of hESMV Treatment

To assess safety, the long-term survival of the animals is determined and examined by histology their eyes, brain, liver, and kidneys for signs of organ damage and tumor formation. The potential immunogenicity of hESMVs within the ocular microenvironment is evaluated using histologic, proteomic and gene expression profiling. The two routes of administration, intravitreal and subretinal, are independently tested. Initial studies are performed on mouse intact eyes that had not been subjected to NMDA injury. The time course of testing from immune reactions is evaluated to investigate both early and late effects.

Uveitis, an influx of inflammatory cells, can occur early, within 24 hr of exposure to foreign proteins, or can take 2 to 3 weeks to develop. Both hESMV-injected and control eyes from a total of 5 to 10 animals per time point (1, 7, 14, and 21 days following injection) are histologically evaluated to ascertain the presence or absence of inflammatory cells and retinal integrity (Caspi et al. (2008) Ophthalmic Res. 40:169-174; Caspi et al. (2010) J. Clin. Invest. 120:3073-3083). The fixation protocol maintains the ability to perform immunohistochemistry of the preserved tissue. If a cellular infiltrate is observed, the eyes are graded in a standard and masked manner, and immunohistochemistry is used to identify the infiltrating cells. CD3, CD20, and CD68 markers are used to confirm whether the cells are of T, B, or macrophage lineage.

In addition to histology, tunnel staining is performed to look for apoptosis of retinal cells. In these studies horizontal sections through the retina adjacent to the optic nerve are stained, analyzed, and quantified using microscopy. If the transfer of human-derived MVs into the mouse introduces a response that is secondary to xenogenicity, these studies are repeated in immunodeficient mice.

In the absence of a cellular inflammatory response eyes exposed to hESMVs are tested using proteomic analysis for proinflammatory molecules and upregulation of genes involved in immunity and inflammation. Whole eyes are prepared for RNA isolation using RNase-free conditions and for protein extraction using a cocktail of protease inhibitors. The uveal tissue and vitreous are isolated, homogenized, and either extracted for RNA using a standard kit or for protein using a specific extraction buffer (50 mM Tris, 150 mM NaCl, 1 mM EDTA, 1% NP-40, 1 mM PMSF, 10 μg/ml leupeptin, 10 μg/ml aprotinin, and 1 mM sodium orthovanadate). These tests are carried out also at 4 time points: 1, 7, 14, and 21 days following hESMV injection. Initially, pools of 4 eyes per group (experimental eyes, control fellow eye, and untreated eyes from animals that were not injected) are used for gene expression analysis on an Affymetrix GeneChip 2.0 ST array; gene expression software is used for evaluation of the results. Mouse cytokine analysis is performed on the protein extracts using a 26-plex Luminex array. This tests the following: eotaxin, G-CSF, GM-CSF, IFN-gamma, IL-10, IL-12p40, IL-12p70, IL-13, IL-17, IL-1-alpha, IL-1-beta, IL-2, IL-3, IL-4, IL-5, IL-6, IL-23, IP-10, KC, MCP-1, MCP-3, MIP-1 alpha, RANTES, TGF-beta, TNF-alpha, and VEGF. In the unexpected event that an immunologic response is elicited, then its pathologic consequence is identified by lymphocyte stimulation studies using immune cells in short term cultures.

### Example 19

### Repair of Damaged Retinas of the rd1 Mouse Model of Human arRP with hESMVs

The rd1 mouse has a retinal degeneration caused by a defect in the Pde6b gene: a C⁻>A mutation in codon 347 of exon 7. Loss of rod photoreceptors begins early in life in the rd1 retina and progresses rapidly. Swelling of mitochondria in the inner segment of rods is seen at postnatal day 8 (P8), followed by disruption of the ordered stack of outer segment discs, which reach a very short maximal length (as compared with normal rods) by P12; photoreceptor nuclei begin to become pyknotic at P10. The wave of rod cell death in the next few days results in a rapid thinning of the retinal outer nuclear layer (ONL) that contains the nuclei of photoreceptors. Most rods have died by P21; only cones (3% of all photoreceptors) remain at this time in the retina and slowly die thereafter.

The therapeutic activity of hESMs in the rd1 mouse is investigated using the delivery method and dose determined in the examples above. The effect of hESMV treatment is tested at different developmental times to determine whether the level of retinal damage influences the hESMV activation of Müller cells. hESMVs are injected at P5, P8, P12, P15, and P20, and retinal functional improvement is reflected by increases in the a-wave (photoreceptor response) amplitude of ERGs recorded 14 and 30 days post-ESMV injections. Results are corroborated by morphological examination of hESMV-treated and rd1 untreated retinas, quantifying the width of the outer nuclear layers at the beginning, during and after degeneration (Danciger et al. (2000) Mammalian Genome 11:422-427) and comparing these numbers with those of ONLs of same age normal mouse retinas to determine the % recovery; and by immunohistochemical identification of the restored cell types.

When at least 30% of the ERG a-wave amplitude is improved and about 30% of photoreceptor cells are replenished in the rd1 mouse retina by hESMV treatment, and 30% of the ERG b-wave amplitude and 30% amacrine and ganglion cells are restored in the NMDA injury mouse model, that hESMV activated Miller cells support any degenerating retina's endogenous capacity for regeneration is determined.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A therapeutic composition comprising human embryonic stem cells in an amount effective to cause ocular neural progenitor cells to regenerate.

2. The therapeutic composition of claim 1, wherein the ocular neural progenitor cells are retinal progenitor cells.

3. The therapeutic composition of claim 2, wherein the ocular neural progenitor cells are microglial cells and Müller cells.

4. A method of obtaining retinal neural cells, comprising treating retinal progenitor cells with an amount of an embryonic stem cell-derived microvesicle (ESMV) fraction effective to cause the retinal progenitor cells to differentiate into retinal neural cells.

5. The method of claim 4, wherein the retinal progenitor cells are microglial and/or Müller cells.

6. The method of claim 4, wherein the retinal progenitor cell differentiation into retinal neural cells is measured by the presence of glutamine synthetase, Gad67, NeuN, Brn3a, and Syntaxin 1a in the treated cells.

7. The method of claim 4, wherein the ESMV fraction comprises human ESMVs.

8. A method of obtaining cells with a retinal stem cell phenotype, comprising treating microglial cells and Müller cells for at least 8 hours with an effective amount of an embryonic stem cell-derived microvesicle (ESMV) fraction, and measuring the level of epidermal growth factor receptor (EGFR) in the treated cells, the level of EGFR in cells with a retinal stem cell phenotype being decreased relative to the level of EGFR in untreated microglial cells and Müller cells.

9. A method of treating an eye pathology in a mammal, comprising administering to the eye of the mammal in need thereof a therapeutically effective amount of an embryonic stem cell-derived microvesicle (ESMV) fraction obtained from mammalian embryonic stem cells.

10. The method of claim 9, wherein the ESMV fraction is administered to the eye by intravitreal, subretinal, or intraocular injection, or by topical administration.

11. The method of claim 9, wherein the ESMV fraction is administered by continuous or bolus release.

12. The method of claim 9, wherein the eye pathology is age-related macular degeneration, myopic degeneration, diabetic retinopathy, glaucoma, the retinitis pigmentosa complex, inherited retinal degeneration, uveitis, dry eye, optic neuropathy, corneal or anterior segment ocular diseases, ocular cicatricial pemphigoid, benign or malignant Mooren's corneal ulcer, or rheumatoid arthritis.

13. The method of claim 12, wherein the eye pathology is glaucoma and the ESMV fraction is administered topically, intraocularly, or by intravitreal injection.

14. The method of claim 12, wherein the eye pathology is age-related macular degeneration (AMD) or photoreceptor/RPE degeneration, and the ESMC fraction is administered by intravitreal, intraocular, or subretinal injection.

15. The method of claim 12, wherein the eye pathology is retinal degeneration, and the ESMV fraction is administered by subretinal injection.

16. The method of claim 12, wherein the eye pathology is dry eye, corneal disease, or anterior segment ocular disease, and the ESMV fraction is administered by topical application.

17. The method of claim 9, wherein the mammal is human, and the ESMV fraction comprises human ESMVs.

18. A method of obtaining transformed retinal neural cells, comprising contacting retinal progenitor cells with an amount of an embryonic stem cell-derived microvesicle (ESMV) fraction effective to transform the retinal progenitor cells.

19. The method of claim 18, wherein the retinal progenitor cells are microglial and/or Müller cells.

20. The method of claim 18, wherein the ESMV fraction comprises human ESMVs.

21. A cultured population of neural cells transformed with an embryonic stem cell-derived microvesicle (ESMV) fraction, the cells having a dedifferentiated progenitor phenotype relative to untransformed microglial cells.

22. The cell population of claim 21, which comprises microglial cells

23. The cell population of claim 21, which comprises Müller cells.

24. The Müller cell population of claim 23, wherein embryonic, early retinal, pluripotency, inducers of retinal regeneration, extracellular matrix-modifying genes and genes regulating cell cycle reentry, de-differentiation, and activation of retinal stem cell phenotype are induced.

25. The Müller cell population of claim 23, wherein the Oct4, Lin28, Klf4, LIF, BMP7, oligo2, FaxN4, Dill, Pax6, Rax, IL6, CSF2, FGF2, IGF2, GDNF, MMP3, Hes1, Notch 1, Notch2, NeuroD1, and genes for calbindin 1, syntaxub 1a, and rhodopsin are up-regulated relative to their expression in untransformed Müller cells.

26. The Müller cell population of claim 23, wherein the DNMT3a, GATA4, and EGFR genes are down-regulated relative to their expression in untransformed Müller cells.

27. The Müller cell population of claim 25, wherein the DNMT3a and GATA4 genes are down-regulated relative to their expression in untransformed Müller cells.


SEQUENCE LISTING (6 sequences)

SEQ ID NO: 1 - DNA, 20 bp, Artificial Sequence
  Description of Artificial Sequence Synthetic primer
gccgggctgggtggattctc

SEQ ID NO: 2 - DNA, 20 bp, Artificial Sequence
  Description of Artificial Sequence Synthetic primer
attggggcggtcggcacagg

SEQ ID NO: 3 - DNA, 20 bp, Artificial Sequence
  Description of Artificial Sequence Synthetic primer
tccagaagagggcgtcagat

SEQ ID NO: 4 - DNA, 20 bp, Artificial Sequence
  Description of Artificial Sequence Synthetic primer
ctttggtcccagcattcagg

SEQ ID NO: 5 - DNA, 23 bp, Artificial Sequence
  Description of Artificial Sequence Synthetic primer
aacaatcgcggcggcccgaggag

SEQ ID NO: 6 - DNA, 22 bp, Artificial Sequence
  Description of Artificial Sequence Synthetic primer
gcctcggcgtgccggccctgcg